(19) United States
(12) Patent Application Publication
(10) Pub. No.: US 2007/0249484 A1
(43) Pub. Date: Oct. 25, 2007

(54) Process for the Photoactivation and use of a Catalyst by an Inverted Two-Stage Procedure

(76) Inventors: Johannes Benkhoff, Basel (CH); Tunja Jung, Rheinfelden-Herten (DE); Andreas Valet, Binzen (DE); Kurt Dietliker, Allschwil (CH); Eugene Valentine Sitzmann, Newark, DE (US)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/631,994

(22) PCT Filed: Jul. 11, 2005
(86) PCT No.: PCT/EP05/53299

§ 371 Date: Jan. 9, 2007

(30) Foreign Application Priority Data
Jul. 21, 2004 (EP) 04103477.8

(51) Int. Cl. B01J 37/34; B01J 31/02; C07D 311/00; C07D 487/00; B01J 35/00

(52) U.S. Cl. 502/5

(57) ABSTRACT

The invention pertains to a process for the application of a specific photolatent catalyst (a) wherein a composition of matter, comprising said catalyst, is subjected to irradiation before being further processed.

The present invention pertains to a process for the photoactivation of a photocatalyst by irradiating a formulation comprising said catalyst before said formulation is further processed, i.e. applied to a substrate.

The curing of formulations comprising photolatent catalysts by means of irradiation is known. Radiation curing so far finds most consideration in areas where radically curable compositions can be applied. A host of photolatent catalysts for these radically curing systems have been developed.

Further the crosslinking of compositions comprising photolatent acids and suitable crosslinking components is known, as well as the cleavage of protective groups by photoactively released acids, e.g. in photoresist technology.

In all of said applications the irradiation and thereby activation of the photolatent catalyst is effected after the application of the formulation to a substrate.

With this process curing of thick coating layers or coatings that are opaque due to the incorporation of pigments, glass fibres or other fillers that absorb or scatter radiation with sufficient through-cure is difficult or even impossible.

Further, the curing of shadowed or poorly exposed areas, in particular on three-dimensional objects, is difficult and in such cases the irradiation unit has to be adapted to the size and form of the object to be coated, which requires sophisticated and expensive lamp designs.

Extensive protective measures are required with regard to environment, health and safety, e.g. particular steps to protect the worker performing the application and curing of said compositions have to be taken.

In EP 1002587a process for applying laquer coatings with a spray gun, wherein irradiation is performed prior to the contact of the laquer composition with the substrate, is proposed. As suitable formulation in said process nearly every known radiation curable system, i.e. radically, acid and base-curable, is named, however without giving any specific example as to how the process really can be carried out and works.

In WO 04/069427 an example for a similar process using a spray-gun wherein the light-source is positioned outside of the nozzle is given, employing a base catalysed curing formulation and a specific photolatent base, i.e. 2-Benzyl-2-(dimethylamino)-1-[3,5-dimethoxyphenyl]-1-butanone.

It has now been found, that the above-mentioned draw-backs can be overcome by employing non-radically photocurable systems, combined with inverting this procedure of application of the formulation, followed by radiation exposure to induce curing and by using specific formulations.

Subject of the present invention is a process for the application of a photolatent catalyst (a), wherein a composition of matter, comprising said catalyst, is subjected to irradiation before being further processed, characterized in that the photolatent catalyst is
(a1) a photolatent acid of the formula VI 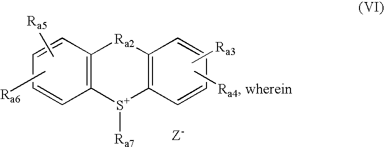
Rₐ₂ is a direct bond, S, O, CH₂, (CH₂)₂, CO or NR₉₆;
Rₐ₃, Rₐ₄, Rₐ₅ and Rₐ₆ independently of one another are H, C₁-C₂₀alkyl, C₃-C₈cycloalkyl, C₁-C₂₀alkoxy, C₂-C₂₀alkenyl, CN, OH, halogen, C₁-C₆alkylthio, phenyl, naphthyl, phenyl-C₁-C₇alkyl, naphtyl-C₁-C₃alkyl, phenoxy, naphthyloxy, phenyl-C₁-C₇alkyloxy, naphtyl-C₁-C₃alkyloxy, phenyl-C₂-C₆alkenyl, naphthyl-C₂-C₄alkenyl, S-phenyl, (CO)Rₐ₈, O(CO)Rₐ₈, (CO)ORₐ₈, SO₂Rₐ₈, OSO₂Rₐ₈;
Rₐ₇ is C₁-C₂₀alkyl, C₁-C₂₀hydroxyalkyl, 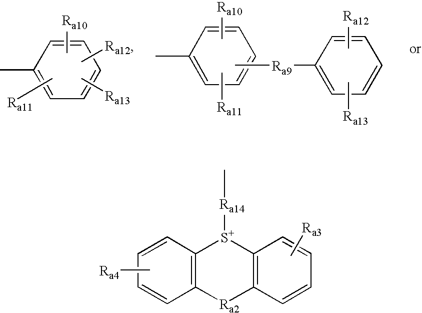
Rₐ₈ is H, C₁-C₁₂alkyl, C₁-C₁₂hydroxyalkyl, phenyl, naphthyl or biphenylyl;
Rₐ₉ is a direct bond, S, O or CH₂;
Rₐ₁₀, Rₐ₁₁, Rₐ₁₂ and Rₐ₁₃ independently of one another have one of the meanings as given for Rₐ₃; or Rₐ₁₀ and Rₐ₁₂ are joined to form a fused ring system with the benzene rings to which they are attached; 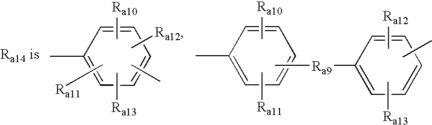
Z is an anion, especially PF₆, SbF₆, AsF₆, BF₄, (C₆F₅)₄B, Cl, Br, HSO₄, CF₃—SO₃, F—SO₃, 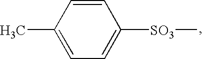
CH₃—SO₃, ClO₄, PO₄, NO₃, SO₄, CH₃—SO₄, H₃C— 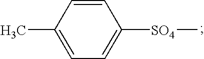
or wherein the photolatent acid (a1) is a compound selected from the group consisting of aromatic phosphonium salts, aromatic iodonium salts or oxime-based photolatent acids; or
   (a2) a photolatent base compound, provided that (3,4-dimethoxy-benzoyl)-1-benzyl-1-dimethylamino propane is excluded, if the composition of matter comprises isocyanates in combination with thiols.

Characterizing for the presently claimed process is the fact, that the activation of the photolatent catalyst by subjecting a composition comprising said catalyst to radiation is carried out prior to the further processing, for example the application to a substrate and that specific formulations and catalysts are employed.

With the process according to the invention one-pack or two-pack formulations can be activated on demand prior to the application without the occurrence of instantaneous curing. The delay time of the formulation until curing starts may be adjusted by modification of the resin components, by the activity of the catalyst or by using adequate inhibitors to a desired level for the requirements of an application process. Since the activation is separated from the application step, any radiation source can be used to activate the formulation independent on the requirements of the application process. For example, with the process according to the invention high energy UV radiation can be used to efficiently set free the catalyst, without the problem of safety issues, which under the conditions of the conventional process in such cases have to be taken. By using high-energy radiation the use of bathochromic-shifted chromophores, which either induce undesired yellowing in the cured film or may be activated by daylight from the environment, can be avoided.

Due to the sequence of relevant process steps in the process according to the invention, between the activation of the catalyst and the curing of the formulation, either before or after application of the coating, other process steps may be introduced as required (e.g. forming of the coated object, build-up of laminate-type structures etc.). With the process according to the invention it is possible to transform conventional 2K systems into easy-to-use 1K systems in the presence of the blocked catalyst, which allows easy handling and extended shelf-life of ready-to-use formulations. Highly reactive 2K systems are currently difficult to handle in industrial processes where downtimes may occur, since after mixing the material crosslinking within the paint equipment immediately starts. This may result in loss of material and/or damage of the equipment if the formulation is gelled during the machine downtime in the application equipment.

With the process according to the invention a broad variety of catalysable formulations can now be conveniently handled and applied as radiation curable systems. Advantages are adjustable reactivity after activation of the catalyst and hence curing at lower temperatures.

Depending on the corresponding composition of matter to be crosslinked or further processed, the photolatent catalyst (a) for example is a photolatent acid compound (a1) or a photolatent base compound (a2). A photolatent acid compound is a compound releasing an acid upon irradiation, while a photolatent base compound is understood to be a compound releasing a base upon irradiation with electromagnetic radiation.

Interesting therefore is a process as described above, wherein
(A) the photolatent catalyst (a) is a photolatent acid (a1) and the composition of matter comprises acid-catalysed curable compounds (b); or wherein
(B) the photolatent catalyst (a) is a photolatent base (a2) and the composition of matter comprises base-catalysed curable compounds (c); or wherein
(C) the photolatent catalyst (a) is a mixture of at least one photolatent base catalyst (a2) and at least one photolatent acid catalyst (a1) and wherein the composition of matter comprises a mixture of acid-catalysed curable compounds (b) and base-catalysed curable compounds (c), provided that (a1) and (a2) are selectively activated.

Suitable photoinitiators (a1) for crosslinking component (b) are e.g. photolatent Lewis and Brønstedt acids, cationic photoinitiators, for example aromatic sulfonium salts, as described for example in WO 03/072567 and WO 03/00840; phosphonium or iodonium salts, such as are described e.g. in U.S. Pat. No. 4,950,581, column 18, line 60 to column 19, line 10, WO 01/09075, WO 98/46647, U.S. Pat. No. 6,306,555 or WO 01/44343; non-ionic photolatent acids, for example photolatent sulfonic acids such as oxime-based photolatent acids, as described, for example, in GB 2348644, U.S. Pat. No. 4,450,598, U.S. Pat. No. 4,136,055, WO 00/10972, WO 00/26219, WO 02/25376, WO 02/98870, WO 03/067332 and WO 04/074242; α-sulfonyloxyketones as described by Berner et al., J. Radiat. Curing 1986, 13(4), 10; α-hydroxymethylbenzoin sulfonates described in EP 89922; ortho-nitrobenzyl sulfonates reported by Houlihan et al. Macromolecules 1988, 21, 2001; 4-nitrobenzyl benzyl esters of sulfonic acids reported by Naitho et al, J. Phys. Chem. 1992, 96, 238, pentafluorobenzyl sulfonates described by Barclay et al. 10^{th} Int. Conf. On Photopolyme., Abstracts Session II PISPE, 1994, aryl diazidonaphthoquinone-4-sulfonates described by Buhr et al. Polym. Mat. Sci. Eng. 1989, 61, 269; α-sulfonyl acetophenones described by LiBassi et al, Conf. Proc. Radcure 86, Apr. 27, 1986, methansulfonate esters of 2-hydroxy or 2,4-dihydroxybenzophenone as reported by Pappas et al, J. Radiat. Curing 1980, 71(1), 2, sulfonate esters of pyrogallol and its analogs described by Ueno et al. Polym. Eng. Sci. 1992, 32, 1511; diaryldilufones described by Aoai et al., J. Photopolym. Sci. Tenol. 1990, 3, 389, N-sulfonyloxyimides as reported by Renner et al. in U.S. Pat. No. 4,371,605, or sulfonated N-hydroxylamines as described in U.S. Pat. No. 4,371,605. Further other types of non-ionic photolatent acids can also be used, such as trichlorormethyltriazine derivatives as described e.g. in EP 332044, α-halogenated acetophenone derivatives described by Peeters et al., Polym. Paint. Colour J. 1989, 179. 304, vicinal dibromides reported by Gannon et al, J. Org. Chem. 1993, 58, 913, triaryl phosphates described by Givens et al. Chem. Rev. 1993, 93, 55, or the ortho-nitrobenzyl triarylsiloxane ethers in combination with aluminium complexes described by Hayase et al, Macromolecules 1985, 18, 1799.

Preferred photolatent acids are, for example, compounds of formula V, VI, VII or/and VIIa 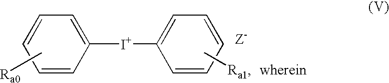
Rₐ₀ and Rₐ₁ are each independently of the other hydrogen, C₁-C₂₀alkyl, C₁-C₂₀alkoxy, OH-substituted C₁-C₂₀alkoxy, halogen, C₂-C₁₂alkenyl, cycloalkyl, especially methyl, isopropyl or isobutyl; and
Z is an anion, especially PF₆, SbF₆, AsF₆, BF₄, (C₆F₅)₄B, Cl, Br, HSO₄, CF₃—SO₃, F—SO₃, 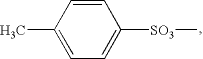
CH₃—SO₃, ClO₄, PO₄, NO₃, SO₄, CH₃—SO₄, 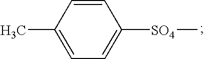 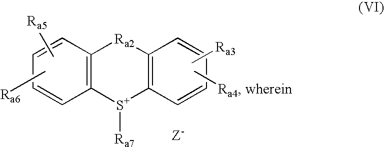
Rₐ₂ is a direct bond, S, O, CH₂, (CH₂)₂, CO or NR₉₆;
Rₐ₃, Rₐ₄, Rₐ₅ and Rₐ₆ independently of one another are H, C₁-C₂₀alkyl, C₃-C₈cycloalkyl, C₁-C₂₀alkoxy, C₂-C₂₀alkenyl, CN, OH, halogen, C₁-C₆alkylthio, phenyl, naphthyl, phenyl-C₁-C₇alkyl, naphtyl-C₁-C₃alkyl, phenoxy, naphthyloxy, phenyl-C₁-C₇alkyloxy, naphtyl-C₁-C₃alkyloxy, phenyl-C₂-C₆alkenyl, naphthyl-C₂-C₄alkenyl, S-phenyl, (CO)Rₐ₈, O(CO)Rₐ₈, (CO)ORₐ₈, SO₂Rₐ₈, OSO₂Rₐ₈;
Rₐ₇ is C₁-C₂₀alkyl, C₁-C₂₀hydroxyalkyl, 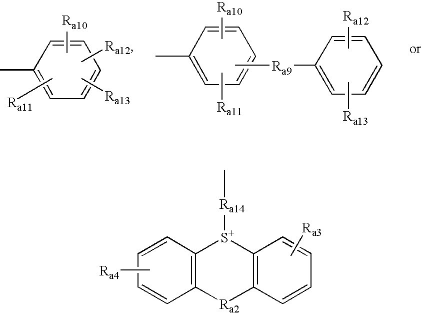
Rₐ₈ is H, C₁-C₁₂alkyl, C₁-C₁₂hydroxyalkyl, phenyl, naphthyl or biphenylyl;
Rₐ₉ is a direct bond, S, O or CH₂;
Rₐ₁₀, Rₐ₁₁, Rₐ₁₂ and Rₐ₁₃ independently of one another have one of the meanings as given for Rₐ₃; or Rₐ₁₀ and Rₐ₁₂ are joined to form a fused ring system with the benzene rings to which they are attached;
Rₐ₁₄ is 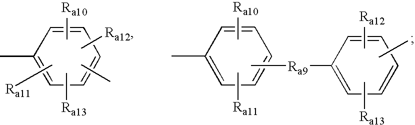
Z is as defined above; 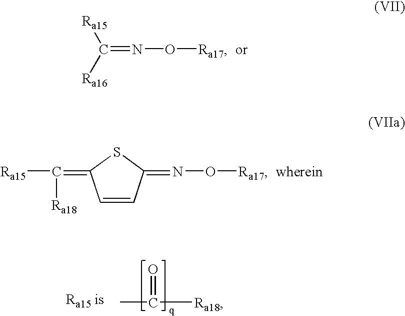
(CO)O—C₁-C₄alkyl, CN or C₁-C₁₂haloalkyl;
Rₐ₁₆ has one of the definitions given for Rₐ₁₅ or is 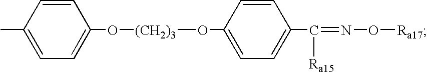
Rₐ₁₇ is C₁-C₁₈alkylsulfonyl, C₁-C₁₀haloalkylsulfonyl, camphorylsulfonyl, phenyl-C₁-C₃alkylsulfonyl, C₃-C₃₀cycloalkylsulfonyl, phenylsulfonyl, naphthylsulfonyl, anthracylsulfonyl or phenanthrylsulfonyl, the groups cycloalkyl, phenyl, naphthyl, anthracyl and phenanthryl of the radicals C₃-C₃₀cycloalkylsulfonyl, phenyl-C₁-C₃alkylsulfonyl, phenylsulfonyl, naphthylsulfonyl, anthracylsulfonyl and phenanthrylsulfonyl being unsubstituted or substituted by one or more halogen, C₁-C₄haloalkyl, CN, NO₂, C₁-C₁₆alkyl, phenyl, C₁-C₄alkylthio, C₁-C₄alkoxy, phenoxy, C₁-C₄alkyl-O(CO)—, C₁-C₄alkyl-(CO)O—, Rₐ₂₇OSO₂— and/or —NRₐ₂₀Rₐ₂₁ substituents; or Rₐ₁₇ is C₂-C₆haloalkanoyl, halobenzoyl, 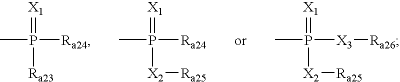
X₁, X₂ and X₃ are each independently of the others O or S;
q is 0 or 1; and
Rₐ₁₈ is C₁-C₁₂alkyl, cyclohexyl, camphoryl, unsubstituted phenyl, or phenyl substituted by one or more halogen, C₁-C₁₂alkyl, ORₐ₁₉, SRₐ₁₉ or NRₐ₂₀Rₐ₂₁ substituents;
Rₐ₁₉ is C₁-C₁₂alkyl, phenyl, phenyl-C₁-C₄alkyl or C₁-C₁₂hydroxyalkyl;
Rₐ₂₀ and Rₐ₂₁ are each independently of the other hydrogen, C₁-C₄alkyl, C₂-C₆hydroxyalkyl, or Rₐ₂₀ and Rₐ₂₁ together with the N atom to which they are bonded, form a 5- or 6-membered ring, which may also contain O atoms or an NRₐ₂ group;
Rₐ₂₂ is hydrogen, phenyl, phenyl-C₁-C₄alkyl, C₁-C₁₂alkyl or C₂-C₅hydroxyalkyl;
Rₐ₂₃, Rₐ₂₄, Rₐ₂₅ and Rₐ₂₆ are each independently of the others C₁-C₆alkyl, C₁-C₆haloalkyl; or phenyl unsubstituted or substituted by C₁-C₄alkyl or by halogen; and
Rₐ₂₇ is hydrogen, C₁-C₄alkyl, phenyl or tolyl.

The specific meanings of the radicals are as described above.

Compounds of formulae V, VI, VII and VIIa are generally known and are in some cases commercially available. Their preparation is known to the person skilled in the art and frequently described in the literature.

Suitable iodonium salts are e.g. tolylcumyliodonium tetrakis(pentafluorophenyl)borate, 4-[(2-hydroxy-tetradecyloxy)phenyl]phenyliodonium hexafluoroantimonate or hexafluorophosphate (SarCat CD 1012; Sartomer), tolylcumyliodonium hexafluorophosphate, 4-isobutylphenyl-4′-methylphenyliodonium hexafluorophosphate (IRGACURE® 250, Ciba Spezialitätenchemie), 4-octyloxyphenyl-phenyliodonium hexafluorophosphate or hexafluoroantimonate, bis(dodecylphenyl)iodonium hexafluoroantimonate or hexafluorophosphate, bis(4-methylphenyl)iodonium hexafluorophosphate, bis(4-methoxyphenyl)iodonium hexafluorophosphate, 4-methylphenyl-4′-ethoxyphenyliodonium hexafluorophosphate, 4-methylphenyl-4′-dodecylphenyliodonium hexafluorophosphate, 4-methylphenyl-4′-phenoxyphenyliodonium hexafluorophosphate. Of all the iodonium salts mentioned, compounds with other anions are, of course, also suitable. The preparation of iodonium salts is known to the person skilled in the art and described in the literature, for example U.S. Pat. No. 4,151,175, U.S. Pat. No. 3,862,333, U.S. Pat. No. 4,694,029, EP 562 897, U.S. Pat. No. 4,399,071, U.S. Pat. No. 6,306,555, WO 98/46647 J. V. Crivello, “Photoinitiated Cationic Polymerization” in: UV Curing: Science and Technology, Editor S. P. Pappas, pages 24-77, Technology Marketing Corporation, Norwalk, Conn. 1980, ISBN No. 0-686-23773-0; J. V. Crivello, J. H. W. Lam, Macromolecules, 10, 1307 (1977) and J. V. Crivello, Ann. Rev. Mater. Sci. 1983, 13, pages 173-190 and J. V. Crivello, Journal of Polymer Science, Part A: Polymer Chemistry, Vol. 37, 4241-4254 (1999).

Preferred iodonium salts are tolylcumyliodonium hexafluorophosphate and 4-isobutylphenyl-4′-methylphenyliodonium hexafluorophosphate.

Suitable oximesulfonates and their preparation can be found, for example, in WO 00/10972, WO 00/26219, GB 2348644, U.S. Pat. No. 4,450,598, WO 98/10335, WO 99/01429, EP 780 729, EP 821 274, U.S. Pat. No. 5,237,059, EP 571 330, EP 241 423, EP 139 609, EP 361 907, EP 199 672, EP48615, EP12158, U.S. Pat. No. 4,136,055, WO 02/25376, WO 02/98870, WO 03/067332 and WO 04/074242.

A summary of further photolatent acid donors is given in the form of a review by M. Shirai and M. Tsunooka in Prog. Polym. Sci., Vol. 21, 1-45 (1996). and in J. Crivello, K. Dietliker, “Photoinititiators for Free Radical Cationic & Anionic Photopolymerisation”, 2^{nd} Edition, Volume III in the Series “Chemistry & Technology of UV & EB Formulation for Coatings, Inks & Paints”, John Wiley/SITA Technology Limited, London, 1998, chapter III (p. 329-463).

Preferred photolatent acids in the method according to the invention are 4-octyloxyphenyl-phenyliodonium hexafluoroantimonate, 4-(2-hydroxy-tetradecyl-1-oxyphenyl)-phenyliodonium hexafluoroantimonate, 4-decyloxyphenyl-phenyliodonium hexafluorophosphate, 4-decylphenyl-phenyl-iodonium hexafluorophosphate, 4-isopropylphenyl-4′-methylphenyliodonium tetra(pentafluorophenyl)borate, 4-isopropylphenyl-4′-methylphenyliodonium hexafluorophosphate, 4-isobutylphenyl-4′-methylphenyliodonium tetra(pentafluorophenyl)borate, 4-isobutylphenyl-4′-methylphenyl-iodonium hexafluorophosphate, in particular tolylcumyliodonium hexafluorophosphate and 4-isobutylphenyl-4′-methylphenyliodonium hexafluorophosphate. Examples of suitable oximesulfonates are α-(methylsulfonyloxyimino)-4-methoxybenzylcyamide, α-(octylsulfonyloxyimino)-4-methoxybenzylcyamide, α-(methylsulfonyloxyimino)-3-methoxybenzylcyamide, α-(methylsulfonyloxyimino)-3,4-dimethylbenzylcyamide, x-(methylsulfonyloxyimino)-thiophene-3-acetonitrile, α-(isopropylsulfonyloxyimino)-thiophene-2-acetonitrile, cis/trans-α-(dodecylsulfonyloxyimino)-thiophene-2-acetonitrile, 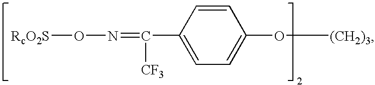
wherein R_{c} is haloalkyl, especially CF₃, and alkyl, especially propyl; 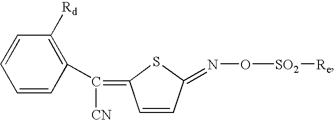
wherein R_{d} is alkyl, especially methyl, and Rₑ
is alkyl, especially methyl, propyl, octyl, camphoryl, p-tolyl or 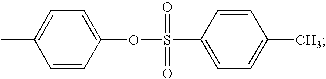
etc. Oxime compounds that yield acids other than sulfonic acids are likewise suitable and are disclosed, for example, in WO 00/26219.

In the context of the present invention, the above list is to be understood as being merely by way of example and on no account as a limitation.

In the process according to the present invention the use of 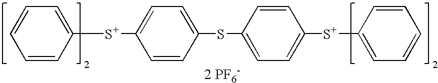
is excluded. In the process according to the present invention the use of (η⁶-isopropylbenzene)(η⁵-cyclopentadienyl)iron(II) hexafluorophosphate is excluded.

In particular interesting is a process, wherein the photolatent acid (a1) is a compound of the formula V, VII or/and VIIa 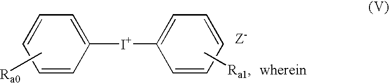
Rₐ₀ and Rₐ₁ are each independently of the other hydrogen, C₁-C₂₀alkyl, C₁-C₂₀alkoxy, OH-substituted C₁-C₂₀alkoxy, halogen, C₂-C₁₂alkenyl, cycloalkyl, especially methyl, isopropyl or isobutyl; and
Z is an anion, especially PF₆, SbF₆, AsF₆, BF₄, (C₆F₅)₄B, Cl, Br, HSO₄, CF₃—SO₃, F—SO₃, 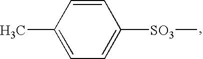
CH₃—SO₃, ClO₄, PO₄, NO₃, SO₄, CH₃—SO₄, 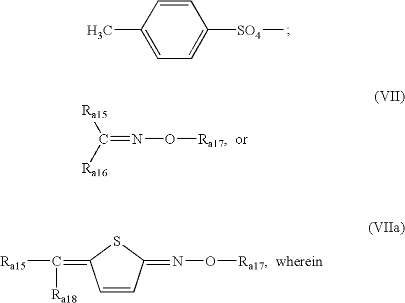
Rₐ₁₅ is 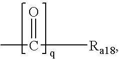
(CO)O—C₁-C₄alkyl, CN or C₁-C₁₂haloalkyl;
Rₐ₁₆ has one of the definitions given for Rₐ₁₅ or is 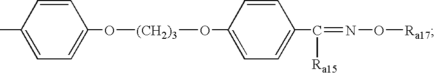
Rₐ₁₇ is C₁-C₁₀alkylsulfonyl, C₁-C₁₀haloalkylsulfonyl, camphorylsulfonyl, phenyl-C₁-C₃alkylsulfonyl, C₃-C₃₀cycloalkylsulfonyl, phenylsulfonyl, naphthylsulfonyl, anthracylsulfonyl or phenanthrylsulfonyl, the groups cycloalkyl, phenyl, naphthyl, anthracyl and phenanthryl of the radicals C₃-C₃₀cycloalkylsulfonyl, phenyl-C₁-C₃alkylsulfonyl, phenylsulfonyl, naphthylsulfonyl, anthracylsulfonyl and phenanthrylsulfonyl being unsubstituted or substituted by one or more halogen, C₁-C₄haloalkyl, CN, NO₂, C₁-C₁₆alkyl, phenyl, C₁-C₄alkylthio, C₁-C₄alkoxy, phenoxy, C₁-C₄alkyl-O(CO)—, C₁-C₄alkyl-(CO)O—, Rₐ₂₇OSO₂— and/or —NRₐ₂₀Rₐ₂₁ substituents; or Rₐ₂₇ is C₂-C₆haloalkanoyl, halobenzoyl, 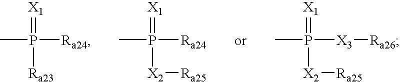
X₁, X₂ and X₃ are each independently of the others O or S;
q is 0 or 1; and
Rₐ₁₈ is C₁-C₁₂alkyl, cyclohexyl, camphoryl, unsubstituted phenyl, or phenyl substituted by one or more halogen, C₁-C₁₂alkyl, ORₐ₁₉, SRₐ₁₉ or NRₐ₂₀Rₐ₂₁ substituents;
Rₐ₁₉ is C₁-C₁₂alkyl, phenyl, phenyl-C₁-C₄alkyl or C₁-C₁₂hydroxyalkyl;
Rₐ₂₀ and Rₐ₂₁ are each independently of the other hydrogen, C₁-C₄alkyl, C₂-C₆hydroxyalkyl, or Rₐ₂₀ and Rₐ₂₁, together with the N atom to which they are bonded, form a 5- or 6-membered ring, which may also contain O atoms or an NRₐ group;
Rₐ₂₂ is hydrogen, phenyl, phenyl-C₁-C₄alkyl, C₁-C₁₂alkyl or C₂-C₅hydroxyalkyl;
Rₐ₂₃, Rₐ₂₄, Rₐ₂₅ and Rₐ₂₆ are each independently of the others C₁-C₆alkyl, C₁-C₆haloalkyl; or phenyl unsubstituted or substituted by C₁-C₄alkyl or by halogen; and
Rₐ₂₇ is hydrogen, C₁-C₄alkyl, phenyl or tolyl.

Preferred photolatent acids in the process according to the present invention are iodonium salts and oximsulfonic acid esters, in particular oximsulfonic acid esters.

In particular preferred as component (a1) are compounds of the formula VII and VIIa.

Another interesting process according to the invention is a process as described above, wherein the photolatent catalyst (a) is a photolatent base (a2) and

the composition of matter comprises base-catalysed curable compounds (c).

As photolatent bases (a2) there come into consideration, for example, capped amine compounds, for example generally the photolatent bases known in the art. Examples are compounds of the classes: o-nitrobenzyloxycarbonylamines, 3,5-dimethoxy-α,α-dimethylbenzyloxycarbonylamines, benzoin carbamates, derivatives of anilides, photolatent guanidines, generally photolatent tertiary amines, for example ammonium salts of α-ketocarboxylic acids, or other carboxylates, benzhydrylammonium salts, N-(benzophenonylmethyl)-tri-N-alkylammonium triphenylalkyl borates, photolatent bases based on metal complexes, e.g. cobalt amine complexes, tungsten and chromium pyridinium pentacarbonyl complexes, anion-generating photoinitators based on metals, such as chromium and cobalt complexes “Reinecke salts” or metalloporphyrins. Examples thereof are published in J. V. Crivello, K. Dietliker “Photoinitiators for Free Radical, Cationic & Anionic Photopolymerisation”, Vol. III of “Chemistry & Technology of UV & EB Formulation for Coatings, Inks & Paints”, 2nd Ed., J. Wiley and Sons/SITA Technology (London), 1998.

Also suitable as photolatent base catalyst for the compositions according to the invention are bases as described in WO 97/31033. They are especially latent bases based on secondary amines, guanidines or amidines. Examples are compounds of formula (A) 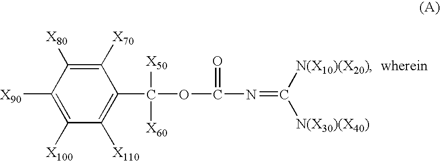
X₁₀, X₂₀, X₃₀, X₄₀, X₅₀, X₆₀, X₇₀, X₈₀, X₉₀, X₁₀₀ and X₁₁₀ are each independently of the others hydrogen, C₁-C₂₀alkyl, aryl, arylalkyl, halogen, alkoxy, aryloxy, arylalkyloxy, aryl-N—, alkyl-N—, arylalkyl-N—, alkylthio, arylthio, arylalkylthio, NO—, CN, a carboxylic acid ester radical, a carboxylic acid amide radical or a ketone or aldehyde radical, or X₁₀, X₂₀, X₃₀ and X₄₀ may form a ring structure and X₅₀, X₆₀, X₇₀, X₈₀, X₉₀, X₁₀₀ and X₁₁₀ independently of X₁₀, X₂₀, X₃₀ and X₄₀ may form one or more further ring structures.

Other suitable photolatent bases are disclosed in EP 764 698. They are capped amino compounds, for example of formula (B) 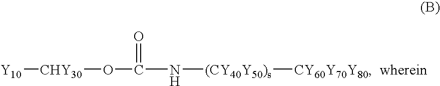
Y₁₀ is a radical 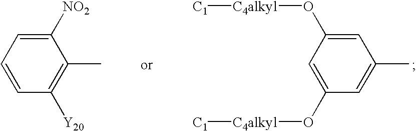
Y₂₀ is hydrogen or NO₂;
Y₃₀ is hydrogen or C₁-C₈alkyl;
Y₄₀, Y₅₀, Y₆₀, Y₇₀ and Y₈₀ are each independently of the others hydrogen or F; and
s is a number from 15 to 29.

It is also possible, to use compounds based on α-aminoketones, as described in EP 898 202 and WO 98/32756, based on α-ammonium, iminium or amidinium ketones and arylborates, as disclosed in WO 98/38195, and based on α-aminoalkenes according to WO 98/41524.

In the compositions according to the invention it is preferred to use compounds from which an amidine group is removed on irradiation with visible light or UV light. They contain a structural element of formula 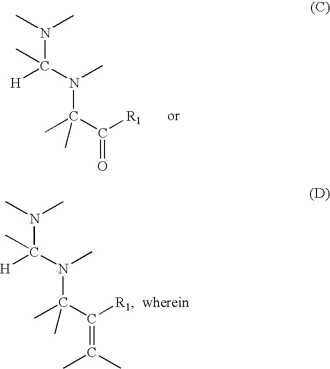
R₁ is an aromatic or heteroaromatic radical capable of absorbing light in the wavelength range from 200 to 650 nm and in doing so brings about cleavage of the adjacent carbon-nitrogen bond.

In particular interesting are compounds of the formula (C1) and (D1) 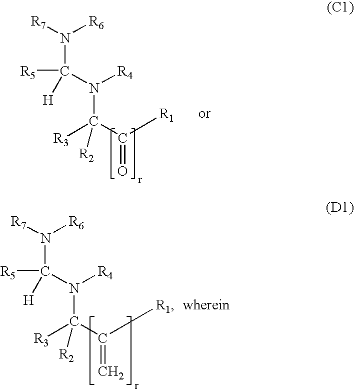
R₁ is an aromatic or heteroaromatic radical which is capable of absorbing light in the wavelength range from 200 to 650 nm and in doing so brings about cleavage of the adjacent carbon-nitrogen bond;
r is 0 or 1;
R₂ and R₃ independently of one another are hydrogen, C₁-C₁₈alkyl, C₃-C₁₈alkenyl, C₃-C₁₈alkynyl or phenyl and, if R₂ is hydrogen or C₁-C₁₈alkyl, R₃ is additionally a group —CO—R₁₄;
or R₁ and R₃, together with the carbonyl group and the C atom to which R₃ is attached, form a benzocyclopentanone radical;
R₅ is C₁-C₁₈alkyl or NR₁₅R₁₆;
R₄, R₆, R₇, R₁ and R₁₆ independently of one another are hydrogen or C₁-C₁₈alkyl; or
R₄ and R₆ together form a C₂-C₁₂alkylene bridge or
R₅ and R₇ together, independently of R₄ and R₆, form a C₂-C₁₂alkylene bridge or, if R₅ is NR₁₅R₁₆, R₁₆ and R₇ together form a C₂-C₁₂alkylene bridge; and
R₁₄ is C₁-C₁₈alkyl or phenyl.

Examples for R₁ as an aromatic or heteroaromatic radical are phenyl, naphthyl, phenanthryl, anthryl, pyrenyl, 5,6,7,8-tetrahydro-2-naphthyl, 5,6,7,8-tetrahydro-1-naphthyl, thienyl, benzo[b]thienyl, naphtho[2,3-b]thienyl, thianthrenyl, dibenzofuryl, chromenyl, xanthenyl, thioxanthyl, phenoxathinyl, pyrrolyl, imidazolyl, pyrazolyl, pyrazinyl, pyrimidinyl, pyridazinyl, indolizinyl, isoindolyl, indolyl, indazolyl, purinyl, quinolizinyl, isoquinolyl, quinolyl, phthalazinyl, naphthyridinyl, quinoxalinyl, quinazolinyl, cinnolinyl, pteridinyl, carbazolyl, β-carbolinyl, phenanthridinyl, acridinyl, perimidinyl, phenanthrolinyl, phenazinyl, isothiazolyl, phenothiazinyl, isoxazolyl, furazanyl, terphenyl, stilbenzyl, fluorenyl or phenoxazinyl, these radicals being un-substituted or mono- or polysubstituted by C₁-C₁₈alkyl, C₃-C₁₈alkenyl, C₃-C₁₈alkynyl, C₁-C₁₈haloalkyl, NO₂, NR₆R₇, N₃, OH, CN, OR₈, SR₈, C(O)R₉, C(O)ORₐ₀ or halogen, or R₁ is a radical of formula A1 or B1 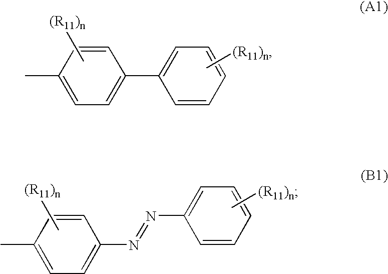
R₆ and R₇ are as defined above;
R₈, R₉, R₁₀, R₁₁ and R₁₂ are hydrogen or C₁-C₁₈alkyl;
R₁₃ is C₁-C₁₈alkyl, C₂-C₁₈alkenyl, C₂-C₁₈alkynyl, C₁-C₁₈haloalkyl, NO₂, NR₈R₉, OH, CN, OR₁₀, SR₁₀, C(O)R₁₁, C(O)OR₁₂ or halogen; and
n is 0 or a number 1, 2 or 3.

Other interesting photolatent base compounds suitable in the process according to the invention are compounds of the formula (C2) and (D2) 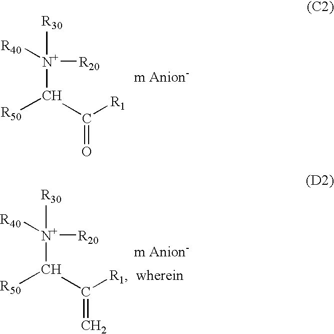
R₁ is as defined above;
R₂₀, R₃₀ and R₄₀ are each independently of one another hydrogen, C₁-C₁₈alkyl, C₃-C₁₈alkenyl, C₃-C₁₈alkynyl or phenyl, or R₂₀ and R₃₀ and/or R₄₀ and R₃₀ form each independently of one another a C₂-C₁₂alkylene bridge; or R₂₀, R₃₀, R₄₀, together with the linking nitrogen atom, are a phosphazene base of the P₁, P₂, P<t/4> type or a group of the structural formula (a), (b), (c), (d), (e), (f) or (g) 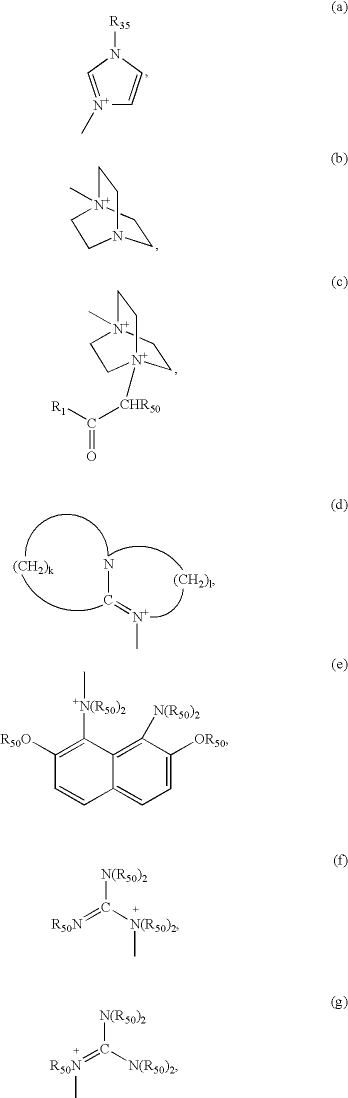
k and l are each independently of the other a number from 2 to 12;
R₃₅ is hydrogen or C₁-C₁₈alkyl;
R₅₀ is hydrogen or C₁-C₁₈alkyl; or
R₅₀ and R₁, together with the linking carbon atoms, are a benzocyclopentanone radical;
R₁₁ is C₁-C₁₈alkyl, C₂-C₁₈alkenyl, C₂-C₁₈alkynyl, C₁-C₁₈haloalkyl, NO₂, NR₆₀R₇₀, OH, CN, OR₈₀, SR₈₀, C(O)R₉₀, C(O)OR₁₀₀ or halogen;
R₆₀, R₇₀, R₈₀, R₉₀ and R₁₀₀ are hydrogen or C₁-C₁₈alkyl;
n is 0 or 1, 2 or 3; and
m is the number of positively charged N-atoms in the molecule.

The “Anion” is any anion capable to form the salt, in particular halogenides, such a Cl, Br or I, borates, such as for example 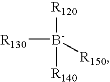
wherein R₁₂₀, R₁₃₀ and R₁₄₀ are phenyl or another aromatic hydrocarbon, these radicals being unsubstituted or mono- or polysubstituted by C₁-C₁₁alkyl, C₃-C₁₈alkenyl, C₃-C₁₈alkynyl, C₁-C₁₈haloalkyl, NO₂, OH, CN, ORB, SR₈, C(O)R₉₀, C(O)OR₁₁₀ or halogen; R₁₅₀ is C₁-C₁₈alkyl, phenyl or another aromatic hydrocarbon, the radicals phenyl and aromatic hydrocarbon being unsubstituted or mono- or polysubstituted by C₁-C₁₈alkyl, C₃-C₁₈alkenyl, C₃-C₁₈alkynyl, C₁-C₁₈haloalkyl, NO₂, OH, CN, OR₈₀, SR₈₀, C(O)R₉₀, C(O)OR₁₀₀ or halogen, or R₁₅₀ is a radical 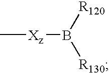 -X_{Z}-B' R₁₂₀; R₁₃₀
or R₁₂₀, R₁₃₀, R₁₄₀ and R₁₅₀ are halogen; and X_{Z} is C₁-C₂₀alkylene, C₂-C₂₀alkylene which is interrupted by —O—, —S— or NR₈₀, or X_{Z} is phenylene, biphenylene, terphenylene, naphthylene, anthrylene or phenylene-CO-phenylene. The Anion further can be one of the Anions as defined above for “Z”, i.e. PF₆, SbF₆, AsF₆, BF₄, (C₆F₅)₄B, Cl, Br, HSO₄, CF₃—SO₃, F—SO₃, 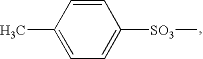
—CH₃—SO₃, ClO₄, PO₄, NO₃, SO₄, CH₃—SO₄,

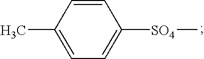

Preferred are compounds of the formula C1 and D1, wherein R₄ and R₆ together form a C₂-C₁₂alkylene bridge and R₅ and R₇ together, independently of R₄ and R₆, form a C₂-C₁₂alkylene bridge.

Preferred are compounds of the formula C2 and D2, wherein R₂₀ is a group of the formula (a), (b) or (c).

Of special interest are photolatent base structures like 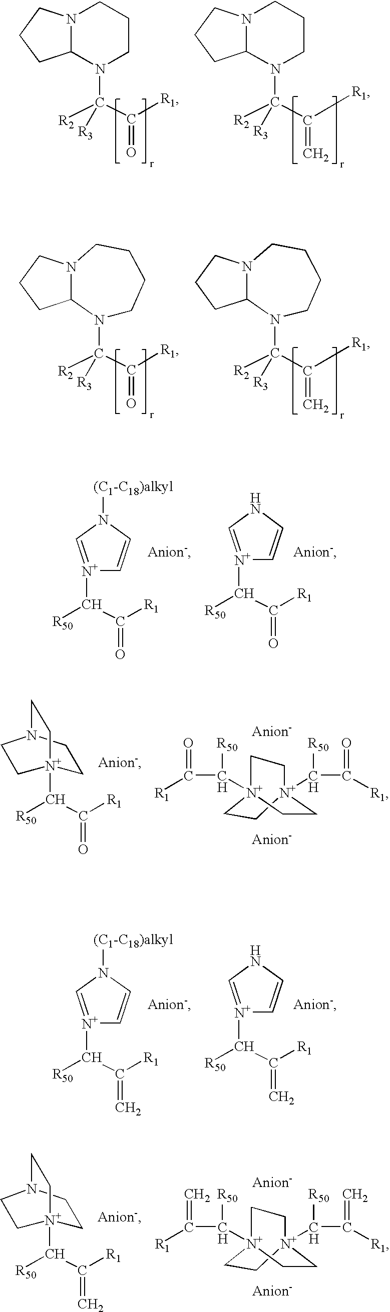
wherein r, R₁, R₂, R₃ and R₅₀ are as defined above.

R₁ preferably is phenyl, naphthyl, pyrenyl, thioxanthyl or penothiazinyl, which radicals are un-substituted or mono- or polysubstituted by C₁-C₁₈alkyl, C₁-C₁₈haloalkyl, NR₈₀R₇₀, CN, NO₂, SR₈₀ or OR₈₀. In particular R₁ is unsubstituted or mono- or polysubstituted phenyl. R₅₀ preferably is hydrogen or C₁-C₄alkyl, in particular hydrogen and methyl.

Preferred photolatent bases are, for example, compounds of formula VIII, VIIIa and VIIIb

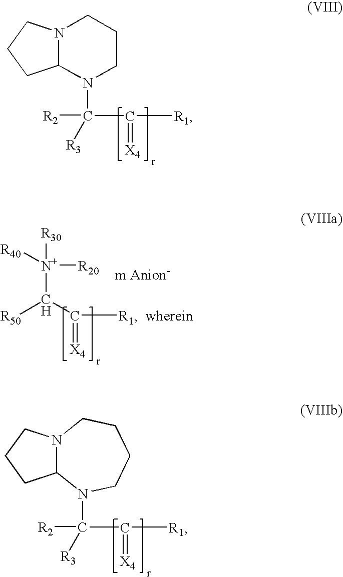

r is 0 or 1;

X₄ is CH₂ or O;

R₂ and R₃ are each independently of the other hydrogen or C₁-C₂₀alkyl;

R₁ is unsubstituted or C₁-C₁₂alkyl- or C₁-C₁₂alkoxy-substituted phenyl, naphthyl or biphenylyl;

R₂₀, R₃₀ and R₄₀, together with the linking nitrogen atom, are a group of the structural formula (a), (b) or (c) 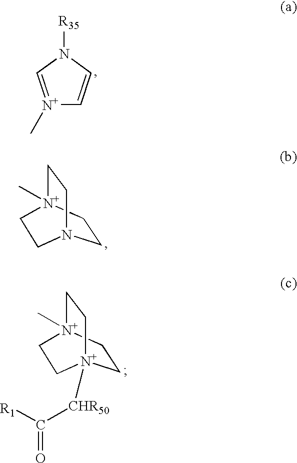
R₃₅ is hydrogen or C₁-C₁₈alkyl;
Anion is any anion capable to form the salt; and
m is the number of positively charged N-atoms in the molecule.

The C₁-C₁₈alkyl in group (a) preferably is methyl.

The preparation of the compounds of formulae (C), (C1), (C2), (D), (D1), (D2), (VIII) and (VIIIa) is known and is described in WO 98/32756, WO 98/38195, WO 98/41524, WO 00/10964 and EP 1436297. Those specifications also provide more specific examples of such compounds, which are incorporated herein by reference. The exact meanings for the definitions of R₁-R₁₆ in formulae C, C1, D, D1 and VIII in the context of the present invention are meant to be like the ones given for the corresponding radicals in WO 98/32756. This teaching is incorporated herein by reference. The exact meanings for the definitions of R₁, R₂₀, R₃₀, R₄₀, R₅₀, R₆₀, R₇₀, R₈₀, R₉₀, R₁₀₀, R₁₂₀, R₁₃₀, R₁₄₀, X_{Z}, groups (a), (b), (c), (d), (e), (f), (g), and the phosphazene bases of the P₁, P₂, P<t/4> type in formulae C2, D2 and VIIIa in the context of the present invention are meant to be like the ones given for the corresponding radicals in WO 00/10964. This teaching is incorporated herein by reference.

Also suitable as photolatent base donors are the α-aminoketone compounds described in EP 898202, for example (4-morpholinobenzoyl)-1-benzyl-1-dimethylamino-propane or (4-methylthiobenzoyl)-1-methyl-1-morpholino-ethane.

In an embodiment of the process according to the invention the use of α-aminoketones as photolatent base is excluded in formulations comprising both, thiols and isocyanates.

In an embodiment of the process according to the invention the use of α-aminoketones as photolatent base is excluded, if the composition of matter is a coating formulation.

In an embodiment of the process according to the invention the use of α-aminoketones as photolatent base is excluded.

In an embodiment of the process according to the invention the use of (3,4-dimethoxybenzoyl)-1-benzyl-1-dimethylamino propane is excluded in formulations comprising both, thiols and isocyanates.

In an embodiment of the process according to the invention the use of (3,4-dimethoxybenzoyl)-1-benzyl-1-dimethylamino propane is excluded, if the composition of matter is a coating formulation.

In an embodiment of the process according to the invention the use of (3,4-dimethoxybenzoyl)-1-benzyl-1-dimethylamino propane is excluded.

Examples of preferred photolatent bases in the method according to the invention are

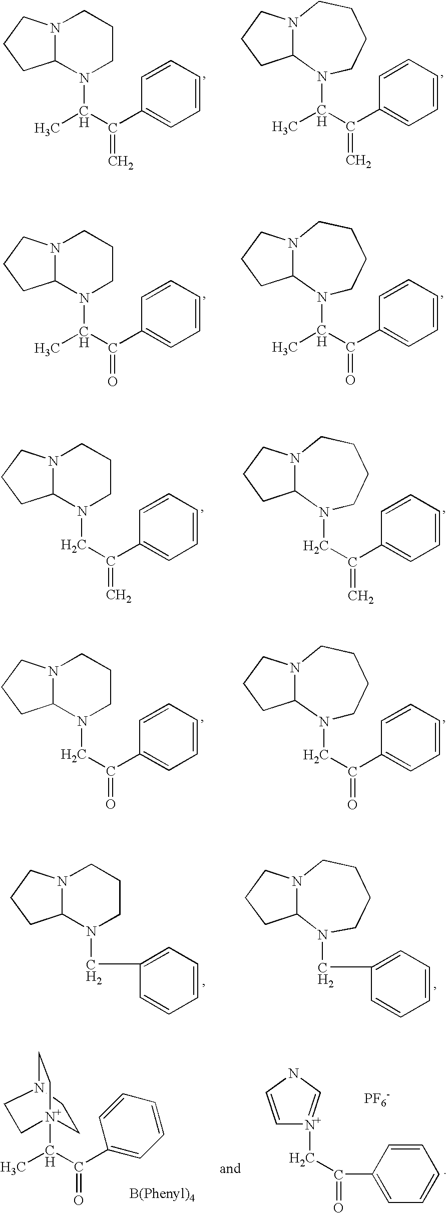

In some cases it may be advantageous to use mixtures of two or more photoinitiators. They may be mixtures of a plurality of photolatent acids, mixtures of a plurality of photolatent bases, and also mixtures of free-radical photoinitiators with photolatent acids (e.g. for use in so-called hybrid systems) or mixtures of free-radical photoinitiators and photolatent bases or mixtures of free-radical photoinitiators with photolatent acids and photolatent bases.

In the context of the present invention, the above list is to be understood as being merely by way of example and on no account as a limitation.

A summary of further photobase generators is given in the form of a review by M. Shirai and M. Tsunooka in Prog. Polym. Sci., Vol. 21, 1-45 (1996). and in J. Crivello, K. Dietliker, “Photoinititiators for Free Radical Cationic & Anionic Photopolymerisation”, 2^{nd} Edition, Volume III in the Series “Chemistry & Technology of UV & EB Formulation for Coatings, Inks & Paints”, John Wiley/SITA Technology Limited, London, 1998, chapter IVI (p. 479-544).

The photopolymerisable compositions comprise the photolatent catalyst (a), i.e. (a1) and/or (a2) advantageously in an amount of from 0.01 to 20% by weight, e.g. from 0.05 to 15% by weight, preferably from 0.1 to 20% by weight, e.g. from 1 to 15% by weight, preferably from 1 to 5% by weight, based on the composition. The given amount of photoinitiator relates to the sum of all added photoinitiators when mixtures thereof are used.

An acid-catalysed curable component (b) is a compound that, under the action of an acid, is able to enter into a polymerisation, polycondensation or polyaddition reaction.

The compositions according to the invention comprise as component (b) e.g. resins and compounds that can be polymerised cationically by alkyl- or aryl-containing cations or by protons. Examples thereof are cyclic ethers, especially epoxides and oxetanes, and also vinyl ethers and hydroxyl-containing compounds. Lactone compounds and cyclic thioethers and also vinyl thioethers can also be used. Further examples are aminoplasts or phenolic resol resins. They are especially melamine, urea, epoxy, phenol, acrylic, polyester and alkyd resins, but more especially mixtures of acrylic, polyester or alkyd resins with a melamine resin. Also included are modified surface-coating resins, e.g. acrylic-modified polyester and alkyd resins. Examples of individual types of resins that are included under the terms acrylic, polyester and alkyd resins are described, for example, in Wagner, Sarx/Lackkunstharze (Munich, 1971), pages 86 to 123 and 229 to 238, or in Ullmann/Encyclopädie der techn. Chemie, 4th edition, Vol. 15 (1978), pages 613 to 628, or Ullmann's Encyclopedia of Industrial Chemistry, Verlag Chemie, 1991, Vol. 18, 360 ff., Vol. A19, 371 ff. The component preferably contains an amino resin (especially when the composition is used as a surface coating). Examples thereof are etherified or non-etherified melamine, urea, guanidine or biuret resins. Acid catalysis is especially important for the curing of surface coatings that contain etherified amino resins, e.g. methylated or butylated melamine resins (N-methoxymethyl- or N-butoxymethylmelamine) or methylated/butylated glycol urils. Amido- and amino resins are described, for example, in Stoye Freitag, Lackharze, Carl Hanser Verlag Munchen 1996, p. 104-126 and novolack resins are described, for example in Stoye Freitag, Lackharze, Carl Hanser Verlag München 1996, p. 150-152.

It is also possible, for example, to use all customary epoxides, such as aromatic, aliphatic or cycloaliphatic epoxy resins. They are compounds having at least one epoxy group, preferably at least two epoxy groups, in the molecule. Examples thereof are the glycidyl ethers and β-methylglycidyl ethers of aliphatic or cycloaliphatic diols or polyols, e.g. those of ethylene glycol, propane-1,2-diol, propane-1,3-diol, butane-1,4-diol, diethylene glycol, polyethylene glycol, polypropylene glycol, glycerol, trimethylolpropane or 1,4-dimethylolcyclohexane or of 2,2-bis(4-hydroxycyclohexyl)propane and N,N-bis(2-hydroxyethyl)aniline; the glycidyl ethers of di- and poly-phenols, for example of resorcinol, of 4,4′-dihydroxyphenyl-2,2-propane, of novolaks or of 1,1,2,2-tetrakis(4-hydroxyphenyl)ethane. Examples are phenyl glycidyl ether, p-tert-butyl glycidyl ether, o-cresyl glycidyl ether, polytetrahydrofuran glycidyl ethers, n-butyl glycidyl ether, 2-ethylhexyl glycidyl ether, C_{12/15}alkyl glycidyl ethers, cyclohexanedimethanol diglycidyl ethers. Further examples are N-glycidyl compounds, e.g. the glycidyl compounds of ethyleneurea, 1,3-propyleneurea or 5-dimethylhydantoin or of 4,4′-methylene-5,5′-tetramethyldihydantoin, or compounds such as triglycidyl isocyanurate.

Further examples of glycidyl ether components (b) used in the method according to the invention are glycidyl ethers of monovalent phenols obtained by reaction of polyvalent phenols with an excess of chlorohydrin, for example epichlorohydrin (e.g. glycidyl ethers of 2,2-bis(2,3-epoxypropoxyphenol)propane. Further examples of glycidyl ether epoxides that can be used in the context of the present invention are described e.g. in U.S. Pat. No. 3,018,262 and in “Handbook of Epoxy Resins” by Lee and Neville, McGraw-Hill Book Co., New York (1967).

A large number of commercially available glycidyl ether epoxides are suitable as component (b), for example glycidyl methacrylate, diglycidyl ethers of bisphenol A, e.g. those available under the trade names EPON 828, EPON 825, EPON 1004 and EPON 1010 from Shell; DER-331, DER-332 and DER-334 from Dow Chemical; 1,4-butanediol diglycidyl ether of phenolformaldehyde novolak, e.g. DEN-431, DEN-438 from Dow Chemical; and resorcinol diglycidyl ether; alkyl glycidyl ethers, for example C₈-C₁₀glycidyl ethers, e.g. HELOXY Modifier 7, C₁₂-C₁₄glycidyl ethers, e.g. HELOXY Modifier 8, butyl glycidyl ether, e.g. HELOXY Modifier 61, cresyl glycidyl ether, e.g. HELOXY Modifier 62, p-tert-butylphenyl glycidyl ether, e.g. HELOXY Modifier 65, polyfunctional glycidyl ethers, for example diglycidyl ether of 1,4-butanediol, e.g. HELOXY Modifier 67, diglycidyl ether of neopentyl glycol, e.g. HELOXY Modifier 68, diglycidyl ether of cyclohexanedimethanol, e.g. HELOXY Modifier 107, trimethylolethane triglycidyl ether, e.g. HELOXY Modifier 44, trimethylolpropanetriglycidyl ether, e.g. HELOXY Modifier 48, polyglycidyl ethers of aliphatic polyols, e.g. HELOXY Modifier 84 (all HELOXY glycidyl ethers are available from Shell).

Also suitable are glycidyl ethers that contain copolymers of acrylic esters, e.g. styrene/glycidyl methacrylate or methyl methacrylate/glycidyl acrylate. Examples are 1:1 styrene/glycidyl methacrylate, 1:1 methyl methacrylate/glycidyl acrylate, 62.5:24:13.5 methyl methacrylate/ethyl acrylate/glycidyl methacrylate.

The polymers of the glycidyl ether compounds may, for example, also contain other functionalities, provided that they do not impair the cationic curing.

Other glycidyl ether compounds suitable as component (b) and commercially available from Vantico are polyfunctional liquid and solid novolak glycidyl ether resins, e.g. PY 307, EPN 1179, EPN 1180, EPN 1182 and ECN 9699.

It will be understood that it is also possible to use as component (b) mixtures of different glycidyl ether compounds.

Glycidyl ethers suitable for component (b) are, for example, compounds of formula XX 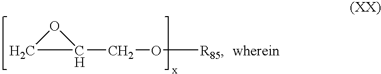
x is a number from 1 to 6; and
R₈₅ is a monovalent to hexavalent alkyl or aryl radical.

Preference is given, for example, to glycidyl ether compounds of formula XX wherein

x is a number 1, 2 or 3; and

R₈₅, when x=1, is unsubstituted or C₁-C₁₂alkyl-substituted phenyl, naphthyl, anthracyl, biphenylyl, C₁-C₂₀alkyl, or C₂-C₂₀alkyl interrupted by one or more oxygen atoms, or

R₈₅, when x=2, is 1,3-phenylene, 1,4-phenylene, C₆-C₁₀cycloalkylene, unsubstituted or halo-substituted C₁-C₄₀alkylene, C₂-C₄₀alkylene interrupted by one or more oxygen atoms, or a group 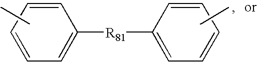
R₈₅, when x=3, is a radical 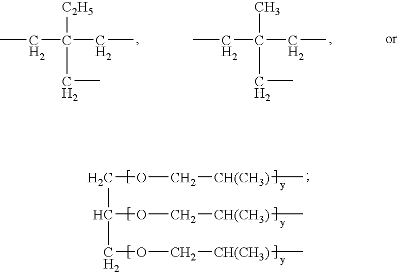
y is a number from 1 to 10; and
R₈₁ is C₁-C₂₀alkylene, oxygen or

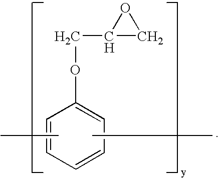

The glycidyl ethers are e.g. compounds of formula XXa 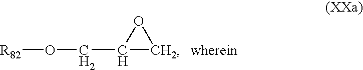
R₈₂ is unsubstituted or C₁-C₁₂alkyl-substituted phenyl; naphthyl; anthracyl; biphenylyl; C₁-C₂₀alkyl, C₂-C₂₀alkyl interrupted by one or more oxygen atoms; or a group of the formula 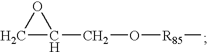
R₈₅ is phenylene, C₁-C₂₀alkylene, C₂-C₂₀alkylene interrupted by one or more oxygen atoms, or a group 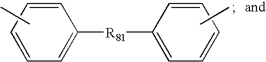
R₈₁ is C₁-C₂₀alkylene or oxygen.

Preference is given to the glycidyl ether compounds of formula XXb 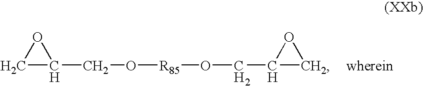
R₈₅ is phenylene, C₁-C₂₀alkylene, C₂-C₂₀alkylene interrupted by one or more oxygen atoms, or a group 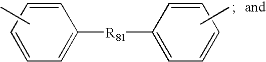
R₈₁ is C₁-C₂₀alkylene or oxygen.

Further examples of component (b) are polyglycidyl ethers and poly(β-methylglycidyl)ethers obtainable by reaction of a compound containing at least two free alcoholic and/or phenolic hydroxyl groups per molecule with the corresponding epichlorohydrin under alkaline conditions, or alternatively in the presence of an acid catalyst with subsequent alkali treatment, it also being possible to use mixtures of different polyols.

Such ethers can be prepared with poly(epichlorohydrin) from acyclic alcohols, such as ethylene glycol, diethylene glycol and higher poly(oxyethylene) glycols, propane-1,2-diol and poly(oxypropylene) glycols, propane-1,3-diol, butane-1,4-diol, poly(oxytetramethylene) glycols, pentane-1,5-diol, hexane-1,6-diol, hexane-2,4,6-triol, glycerol, 1,1,1-trimethylolpropane, pentaerythritol and sorbitol, from cycloaliphatic alcohols, such as resorcitol, quinitol, bis(4-hydroxycyclohexyl)methane, 2,2-bis(4-hydroxycyclohexyl)propane and 1,1-bis(hydroxymethyl)cyclohex-3-ene, and from alcohols having aromatic nuclei, such as N,N-bis(2-hydroxyethyl)aniline and p,p′-bis(2-hydroxyethylamino)diphenylmethane. They can also be prepared from mononuclear phenols, such as resorcinol and hydroquinone, and from polynuclear phenols, such as bis(4-hydroxyphenyl)methane, 4,4-dihydroxydiphenyl, bis(4-hydroxyphenyl)sulfone, 1,1,2,2-tetrakis(4-hydroxyphenyl)ethane, 2,2-bis(4-hydroxyphenyl)propane (bisphenol A) and 2,2-bis(3,5-dibromo-4-hydroxyphenyl)propane.

Further hydroxy compounds suitable for the preparation of polyglycidyl ethers and poly-(β-methylglycidyl)ethers are the novolaks obtainable by condensation of aldehydes, such as formaldehyde, acetaldehyde, chloral and furfural, and phenols, for example phenol, o-cresol, m-cresol, p-cresol, 3,5-dimethylphenol, 4-chlorophenol and 4-tert-butylphenol.

Poly(N-glycidyl) compounds can be obtained, for example, by dehydrochlorination of the reaction products of epichlorohydrin with amines containing at least two amine hydrogen atoms, such as aniline, n-butylamine, bis(4-aminophenyl)methane, bis(4-aminophenyl)propane, bis(4-methylaminophenyl)methane and bis(4-aminophenyl)ether, sulfone and sulfoxide. Further suitable poly(N-glycidyl) compounds are triglycidyl isocyanurate and N,N′-diglycidyl derivatives of cyclic alkyleneureas, such as ethyleneurea and 1,3-propyleneurea, and hydantoins, such as 5,5-dimethylhydantoin.

Poly(S-glycidyl) compounds are also suitable. Examples thereof are the di-S-glycidyl derivatives of dithiols, such as ethane-1,2-dithiol and bis(4-mercaptomethylphenyl)ether.

Also coming into consideration as component (b) are epoxy resins wherein the glycidyl groups or β-methylglycidyl groups are bonded to different kinds of hetero atoms, for example the N,N,O-triglycidyl derivative of 4-aminophenol, the glycidyl ether glycidyl ester of salicylic acid or p-hydroxybenzoic acid, N-glycidyl-N′-(2-glycidyloxypropyl)-5,5-dimethylhydantoin and 2-glycidyloxy-1,3-bis(5,5-dimethyl-1-glycidylhydantoin-3-yl)propane.

Diglycidyl ethers of bisphenols are preferred. Examples thereof are bisphenol A diglycidyl ether, e.g. ARALDIT GY 250 from Huntsman, bisphenol F diglycidyl ether and bisphenol S diglycidyl ether. Special preference is given to bisphenol A diglycidyl ether.

Further glycidyl compounds of technical importance and suitable for use in component (b) are the glycidyl esters of carboxylic acids, especially di- and poly-carboxylic acids. Examples thereof are the glycidyl esters of succinic acid, adipic acid, azelaic acid, sebacic acid, phthalic acid, terephthalic acid, tetra- and hexa-hydrophthalic acid, isophthalic acid or trimellitic acid, or of dimerised fatty acids.

Examples of polyepoxides that are not glycidyl compounds are the epoxides of vinylcyclohexane and dicyclopentadiene, 3-(3′,4′-epoxycyclohexyl)-8,9-epoxy-2,4-dioxaspiro[5,5]-undecane, the 3′,4′-epoxycyclohexylmethyl ester of 3,4-epoxycyclohexanecarboxylic acid, (3,4-epoxycyclohexyl-methyl-3,4-epoxycyclohexanecarboxylate), butadiene diepoxide or isoprene diepoxide, epoxidised linoleic acid derivatives and epoxidised polybutadiene.

Further suitable epoxy compounds are e.g. limonene monoxide, epoxidised soybean oil, bisphenol A and bisphenol F epoxy resins, e.g. Araldit® GY 250 (A), Araldit® GY 282 (F), Araldit® GY 285 (F) (Huntsman), and also photocrosslinkable siloxanes that contain epoxy groups.

Further suitable cationically polymerisable or crosslinkable components (b) can be found e.g. in U.S. Pat. Nos. 3,117,099, 4,299,938 and 4,339,567.

From the group of aliphatic epoxides there are suitable e.g. especially the monofunctional α-olefin epoxides having an unbranched chain consisting of 10, 12, 14 or 16 carbon atoms.

Because a large number of different epoxy compounds are commercially available nowadays, the properties of the binder can vary widely. One possible variation, for example depending upon the intended use of the composition, is the use of mixtures of different epoxy compounds and the addition of flexibilisers and reactive diluents.

The epoxy resins can be diluted with a solvent to facilitate application, for example when application is effected by spraying, but it is preferable to use the epoxy compound in the solventless state. Resins that are viscous to solid at room temperature can be applied, for example, in the hot state.

Also suitable as component (b) are all customary vinyl ethers, such as aromatic, aliphatic or cycloaliphatic vinyl ethers and also silicon-containing vinyl ethers. They are compounds having at least one vinyl ether group, preferably at least two vinyl ether groups, in the molecule. Examples of vinyl ethers that are suitable for use in the method according to the invention are triethylene glycol divinyl ether, 1,4-cyclohexanedimethanol divinyl ether, 4-hydroxybutyl vinyl ether, the propenyl ether of propylene carbonate, dodecylvinyl ether, tert-butyl vinyl ether, tert-amyl vinyl ether, cyclohexyl vinyl ether, 2-ethylhexyl vinyl ether, ethylene glycol monovinyl ether, butanediol monovinyl ether, hexanediol monovinyl ether, 1,4-cyclohexanedimethanol monovinyl ether, diethylene glycol monovinyl ether, ethylene glycol divinyl ether, ethylene glycol butylvinyl ether, butanediol-1,4-divinyl ether, hexanediol divinyl ether, diethylene glycol divinyl ether, triethylene glycol divinyl ether, triethylene glycol methylvinyl ether, tetraethylene glycol divinyl ether, Pluriol-E-200 divinyl ether, polytetrahydrofuran divinyl ether 290, trimethylolpropane trivinyl ether, dipropylene glycol divinyl ether, octadecylvinyl ether, (4-cyclohexyl-methyleneoxyethene)glutaric acid methyl ester and (4-butyloxyethene)isophthalic acid ester.

Examples of hydroxyl-containing compounds are polyester polyols, e.g. polycaprolactones or polyester adipate polyols, glycols and polyether polyols, castor oil, hydroxy-functional vinyl and acrylic resins, cellulose esters, e.g. cellulose acetate butyrate, and phenoxy resins.

Further suitable cationically curable formulations can be found e.g. in EP 119 425.

Preferred as component (b) are cycloaliphatic epoxides, or epoxides based on bisphenol A.

1. surface-coatings based on cold- or hot-crosslinkable alkyd, acrylate, polyester, epoxy, urea or melamine resins or mixtures of such resins, optionally with the addition of a curing catalyst;

2. one-component polyurethane surface-coatings based on aliphatic or aromatic urethane acrylates or polyurethane acrylates having free amine groups in the urethane structure and melamine resins or polyether resins, optionally with the addition of a curing catalyst;

3. thermoplastic polyacrylate surface-coatings based on thermoplastic acrylate resins or extrinsically crosslinking acrylate resins in combination with etherified melamine resins;

4. surface-coatings based on acrlyate resins modified by fluoro or siloxanes;

5. surface-coatings, in particular clear surface-coatings, based malonate-blocked isocyanates with melamine resins (e.g. hexamethoxymethylmelamine) as crosslinker (acid-catalysed);

6. dual-cure systems, that first are cured thermally and afterwards by UV-irradiation—or vice versa—wherein the components of the surface-coating composition comprise double bonds, which are brought to react by UV-light and photoinitiators and/or by electron beam irradiation.

In particular interesting as acid-catalysed curable components (b) are

1. surface-coatings based on cold- or hot-crosslinkable alkyd combined urea or melamine resins, with the addition of a curing catalyst;

2. surface-coatings based on cold- or hot-crosslinkable hydroxyfunctional acrylate and/or polyester combined with urea or melamine resins, with the addition of a curing catalyst;

3. surface-coatings based on cold- or hot-crosslinkable epoxy combined with urea or melamine resins, with the addition of a curing catalyst.

A base-catalysed curable component (c) is a compound that, under the action of a base, is able to enter into a polymerisation, polycondensation or polyaddition reaction.

The base-catalysed polymerisation, addition, condensation or substitution reaction can be carried out with low molecular weight compounds (monomers), with oligomers, with polymeric compounds or with a mixture of such compounds. Examples of reactions that can be carried out either with monomers or with oligomers/polymers using the method according to the invention are the Knoevenagel reaction or Michael addition. The presence of further components may be advantageous or necessary for the reaction. This is disclosed, for example, in EP 1 092 757.

Of special importance are compositions wherein component (c) is an anionically polymerisable or crosslinkable organic material.

The anionically polymerisable or crosslinkable organic material [component (c)] can be in the form of mono- or poly-functional monomers, oligomers or polymers.

Especially preferred oligomeric/polymeric systems (c) are binders customary in the coating industry.

Two-component systems of an α,β-ethylenically unsaturated carbonyl compound and a polymer containing activated CH₂ groups, the activated CH₂ groups being present either in the main chain or in the side chain or in both, as described, for example, in EP 161 697 for (poly)malonate groups. The malonate group can in a polyurethane, polyester, polyacrylate, epoxy resin, polyamide or polyvinyl polymer be bonded either in the main chain or in a side chain. The α,β-ethylenically unsaturated carbonyl compound used may be any double bond activated by a carbonyl group. Examples are esters or amides of acrylic acid or methacrylic acid. Additional hydroxyl groups may also be present in the ester groups. Di- and tri-esters are also possible. Typical examples are hexanediol diacrylate and trimethylolpropane triacrylate. Instead of acrylic acid it is also possible to use other acids and esters or amides thereof, for example crotonic acid or cinnamic acid.

Other compounds having activated CH₂ groups are (poly)acetoacetates and (poly)cyanoacetates.

Further examples are two-component systems of a polymer containing activated CH₂ groups, the activated CH₂ groups being present either in the main chain or in the side chain or in both, or a polymer containing activated CH₂ groups, such as (poly)acetoacetates and (poly)cyanoacetates, and a polyaldehyde crosslinking agent, for example terephthalic aldehyde. Such systems are described, for example, in Urankar et al., Polym. Prepr. (1994), 35, 933.

The components of the system react with one another, with base catalysis, at room temperature and form a crosslinked coating system suitable for many applications. By virtue of its already good resistance to weathering, the system is also suitable, for example, for outdoor applications and can, if necessary, be additionally stabilised by UV absorbers and other light stabilisers.

Also coming into consideration as component (c) in the compositions according to the invention are epoxy systems. Epoxy resins suitable for the preparation of curable mixtures according to the invention having epoxy resins as component (c) are those customary in epoxy resin technology. Examples of such epoxy resins are described above under component (b). Suitable examples are especially polyglycidyl and poly(β-methylglycidyl) esters, obtainable by reaction of a compound having at least two carboxyl groups in the molecule and epichlorohydrin and β-methylepichlorohydrin, respectively. The reaction is advantageously carried out in the presence of bases.

An aliphatic polycarboxylic acid may be used as the compound having at least two carboxyl groups in the molecule. Examples of such polycarboxylic acids are oxalic acid, succinic acid, glutaric acid, adipic acid, pimelic acid, suberic acid, azelaic acid and dimerised or trimerised linoleic acid. It is also possible, however, to use cycloaliphatic polycarboxylic acids, for example tetrahydrophthalic acid, 4-methyltetrahydrophthalic acid, hexahydrophthalic acid or 4-methylhexahydrophthalic acid. Aromatic polycarboxylic acids, for example phthalic acid, isophthalic acid or terephthalic acid, may also be used.

Polyglycidyl or poly(β-methylglycidyl)ethers, obtainable by reaction of a compound having at least two free alcoholic hydroxy groups and/or phenolic hydroxy groups with epichlorohydrin or β-methylepichlorohydrin under alkaline conditions or in the presence of an acid catalyst with subsequent alkali treatment.

The glycidyl ethers of this kind are derived, for example, from acyclic alcohols, such as ethylene glycol, diethylene glycol or higher poly(oxyethylene) glycols, propane-1,2-diol or poly(oxypropylene) glycols, propane-1,3-diol, butane-1,4-diol, poly(oxytetramethylene) glycols, pentane-1,5-diol, hexane-1,6-diol, hexane-2,4,6-triol, glycerol, 1,1,1-trimethylolpropane, pentaerythritol, sorbitol, and also from polyepichlorohydrins. They may also, however, be derived e.g. from cycloaliphatic alcohols, such as 1,4-cyclohexanedimethanol, bis(4-hydroxycyclohexyl)methane or 2,2-bis(4-hydroxycyclohexyl)propane, or they have aromatic nuclei, such as N,N-bis(2-hydroxyethyl)aniline or p,p′-bis(2-hydroxyethylamino)diphenylmethane. The glycidyl ethers can also be derived from mononuclear phenols, for example resorcinol or hydroquinone, or they are based on polynuclear phenols, for example bis(4-hydroxyphenyl)methane, 4,4′-dihydroxybiphenyl, bis(4-hydroxyphenyl)sulfone, 1,1,2,2-tetrakis(4-hydroxyphenyl)ethane, 2,2-bis(4-hydroxyphenyl)propane, 2,2-bis(3,5-dibromo-4-hydroxyphenyl)propane, and on novolaks, obtainable by condensation of aldehydes, such as formaldehyde, acetaldehyde, chloral or furfuraldehyde, with phenols, such as phenol, or with phenols that are substituted in the nucleus by chlorine atoms or by C₁-C₉alkyl groups, e.g. 4-chlorophenol, 2-methylphenol or 4-tert-butylphenol, or by condensation with bisphenols, such as those of the above-mentioned kind.

Poly(N-glycidyl) compounds, obtainable by dehydrochlorination of the reaction products of epichlorohydrin with amines containing at least two amine hydrogen atoms. Such amines are, for example, aniline, n-butylamine, bis(4-aminophenyl)methane, m-xylylenediamine or bis(4-methylaminophenyl)methane.

The poly(N-glycidyl) compounds also include, however, triglycidyl isocyanurate, N,N′-diglycidyl derivatives of cycloalkyleneureas, such as ethyleneurea or 1,3-propyleneurea, and diglycidyl derivatives of hydantoins, such as of 5,5-dimethylhydantoin.

Poly(S-glycidyl) compounds, for example di-S-glycidyl derivatives, derived from dithiols, e.g. ethane-1,2-dithiol or bis(4-mercaptomethylphenyl)ether.

Cycloaliphatic epoxy resins, for example bis(2,3-epoxycyclopentyl)ether, 2,3-epoxycyclopentylglycidyl ether, 1,2-bis(2,3-epoxycyclopentyloxy)ethane or 3,4-epoxycyclohexylmethyl-3′,4′-epoxycyclohexanecarboxylate.

It is also possible, however, to use epoxy resins wherein the 1,2-epoxy groups are bonded to different hetero atoms or functional groups; such compounds include, for example, the N,N,O-triglycidyl derivative of 4-aminophenol, the glycidyl ether glycidyl ester of salicylic acid, N-glycidyl-N′-(2-glycidyloxypropyl)-5,5-dimethylhydantoin and 2-glycidyloxy-1,3-bis(5,5-dimethyl-1-glycidylhydantoin-3-yl)propane.

It is also possible to use mixtures of epoxy resins as component (c). The invention therefore also relates to compositions comprising an epoxy resin or a mixture of different epoxy resins as component (c).

Component (c) may also comprise compounds that are converted into a different form by the action of bases. They are, for example, compounds that, when base-catalysed, e.g. by removal of protecting groups, change their solubility in suitable solvents

As will be seen from the above description, some monomers, oligomers and polymers are suitable as component (b) or (c), because they are both free-radical-crosslinkable and acid- or base-crosslinkable. For example, the two-component systems (2K systems) described above as base-catalysed curable components can also be crosslinked by the addition of a free-radical-forming photoinitiator.

Further interesting components (c) are

1. two-component polyurethane surface-coatings based on hydroxyl-group-containing acrylate, polyester or polyether resins and aliphatic or aromatic isocyanates, isocyanurates or polyisocyanates;

2. two component polyurethane surface-coatings based on thiol group-containing acrylate-, polyester- or polyether resins and aliphatic or aromatic isocyanates, isocyanurates or polyisocyanates;

3. one-component polyurethane surface-coatings based on blocked isocyanates, isocyanurates or polyisocyanates, which are de-blocked during heating; it is also possible to add melamine resins as appropriate;

4. two-component surface-coatings based on (poly)ketimines and aliphatic or aromatic isocyanates, isocyanurates or polyisocyanates;

5. two-component surface-coatings based on (poly)ketimines and an unsaturated acrylate resin or a polyacetoacetate resin or a methacrylamidoglycolate methyl ester;

6. two-component surface-coatings based on carboxyl- or amine group-containing polyacrylates and polyepoxides, in particular based on carboxyl group-containing polyacrylates and polyepoxides;

7. two-component surface-coatings based on anhydride-group-containing acrylate resins and a polyhydroxy or polyamine component, in particular a polyhydroxy component;

8. two-component surface-coatings based on acrylate-containing anhydrides and polyepoxides;

9. two-component surface-coatings based on (poly)oxazolines and anhydride-group-containing acrylate resins or unsaturated acrylate resins or aliphatic or aromatic isocyanates, isocyanurates or polyisocyanates;

10. two-component surface-coatings based on unsaturated (poly)acrylates and (poly)malonates;

11. one component epoxid resin surface-coatings

12. two component surface coatings based on thiol group-containing acrylate-polyester or polyether resins and polyepoxides

13. dual-cure systems that first crosslink thermally via base catalysis and subsequently are cured by UV-irradiation—or vice versa—wherein the components of the surface-coating composition comprise double bonds that are brought to reaction by UV-light and photoinitiators and/or by electron beam irradiation.

In particular interesting as base-catalysed curable components (c) are

1. two-component polyurethane surface-coatings based on hydroxyl-group-containing acrylate, polyester or polyether resins and aliphatic or aromatic isocyanates, isocyanurates or polyisocyanates;

2. two component polyurethane surface-coatings based on thiol group-containing acrylate-, polyester- or polyether resins and aliphatic or aromatic isocyanates, isocyanurates or polyisocyanates;

3. two-component surface-coatings based on carboxyl- or amine group-containing polyacrylates and polyepoxides, in particular based on carboxyl group-containing polyacrylates and polyepoxides;

4. one component epoxid resin surface-coatings

two component surface coatings based on thiol group-containing acrylate-polyester or polyether resins and polyepoxides.

It is also possible in the process according to the invention as the composition of matter, to use a mixture of (b) and (c) and also to employ a mixture of photolatent catalysts (a1) and (a2), provided that the catalysts are activated by irradiation of different wavelengths (i.e. are selectively activated).

Interesting is a process as described above, wherein

the photolatent catalyst (a) is a mixture of at least one photolatent base catalyst (a2) and at least one photolatent acid catalyst (a1) and wherein

the composition of matter comprises a mixture of acid-catalysed curable compounds (b) and base-catalysed curable compounds (c), provided that (a1) and (a2) are selectively activated.

Additionally, radically polymerizable components can be present in the composition of matter according to the present invention. Examples are

(Meth)acrylate compounds which may be mentioned are (meth)acrylic esters, and especially acrylic esters of poly-functional alcohols, especially those comprising, in addition to the hydroxyl groups, either no other functional groups or just ether groups. Examples of such alcohols are bifunctional alcohols, such as ethylene and propylene glycol, and members of the same class with higher degrees of condensation, such as diethylene, triethylene, dipropylene and tripropylene glycol, etc., butanediol, pentanediol, hexanediol, neopentylglycol, alkoxylated phenolic compounds, such as ethoxylated and propoxylated bisphenols, cyclohexane-dimethanol, alcohols with a functionality of three or more, such as glycerol, trimethylolpropane, butanetriol, trimethylolethane, pentaerythritol, ditrimethylolpropane, dipentaerythritol, sorbitol, mannitol and the corresponding alkoxylated alcohols, especially ethoxylated and propoxylated alcohols.

The alkoxylation products can be obtained in a known manner by reacting the above mentioned alcohols with alkylene oxides, especially ethylene or propylene oxide. The degree of alkoxylation per hydroxyl group is preferably 0-10; in other words, 1 mol of hydroxyl group can preferably be alkoxylated with up to 10 mol of alkylene oxides.

Further (meth)acrylate compounds are polyester (meth)acrylates, which are the acrylic esters of polyesterols.

Examples of suitable polyesterols are those as can be prepared by esterification of polycarboxylic acids, preferably dicarboxylic acids, with polyols, preferably diols. The starting materials for hydroxyl-containing polyesters of this kind are known to the person skilled in the art. As dicarboxylic acids preferably employed are succinic, glutaric, adipic, sebacic and ophthalic acid, their isomers and hydrogenation products, and esterifiable derivatives, such as anhydrides or dialkyl esters of said acids. Suitable polyols are the abovementioned alcohols, preferably ethylene glycol, 1,2- and 1,3-propylene glycol, 1,4-butanediol, 1,6-hexanediol, neopentyl glycol, cyclohexane-dimethanol and polyglycols of the ethylene glycol and propylene glycol type.

Polyester (meth)acrylates can be prepared in a plurality of stages or else in one stage, as described for example in EP 279303, from acrylic acid, polycarboxylic acid and polyol.

Other examples are epoxy or urethane (meth)acrylates.

Examples of epoxy(meth)acrylates are those obtainable by reacting epoxidized olefins or poly- and/or diglycidyl ethers, such as bisphenol A diglycidyl ether with (meth)acrylic acid.

The reaction is known to the person skilled in the art and is described, for example, in R. Holmann, U. V. and E. B. Curing Formulation for Printing Inks and Paints. London 1984. Urethane (meth)acrylates are, in particular, reaction products of hydroxyalkyl(meth)acrylates with poly- and/or diisocyanates (see again R. Holmann, U. V and E. B. Curing Formulation for Printing Inks and Paints. London 1984).

It is of course also possible to employ mixtures of different radically polymerizable compounds as described above, including in particular mixtures of the above (meth)acrylic compounds.

The photopolymerisable mixtures may comprise, in addition to the photolatent catalyst (a), various additives (h).

Examples thereof are thermal inhibitors, which are intended to prevent premature polymerisation, e.g. hydroquinone, hydroquinone derivatives, p-methoxyphenol, β-naphthol or sterically hindered phenols, e.g. 2,6-di(tert-butyl)-p-cresol, or 4-hydroxy-2,2,6,6-tetramethyl-piperidin-1-oxyl(p-hydroxy-tempo), bis(2,2,6,6-tetramethyl-1-oxyl-4-piperidinyl)-sebacate and 1-methyl-8-(2,2,6,6-tetramethyl-1-oxyl-4-piperidinyl)-sebacate. In order to increase dark storage stability it is possible to use, for example, copper compounds, such as copper naphthenate, stearate or octoate, phosphorus compounds, for example triphenylphosphine, tributylphosphine, triethyl phosphite, triphenyl phosphite or tribenzyl phosphite, quaternary ammonium compounds, e.g. tetramethylammonium chloride or trimethylbenzylammonium chloride, or hydroxylamine derivatives, e.g. N-diethylhydroxylamine.

As further additives light stabilizers (e) can be added to the compositions. Examples are UV absorbers, e.g. those of the hydroxyphenylbenzotriazole, hydroxyphenylbenzophenone, oxalic acid amide or hydroxyphenyl-s-triazine type. Such compounds can be used on their own or in the form of mixtures, with or without the use of sterically hindered amines (HALS).

Specific examples of such UV absorbers and light stabilisers (e) are

1. 2-(2′-Hydroxyphenyl)-benzotriazoles, e.g. 2-(2′-hydroxy-5′-methylphenyl)-benzotriazole, 2-(3′,5′-di-tert-butyl-2′-hydroxyphenyl)-benzotriazole, 2-(5′-tert-butyl-2′-hydroxyphenyl)-benzotriazole, 2-(2′-hydroxy-5′-(1,1,3,3-tetramethylbutyl)-phenyl)-benzotriazole, 2-(3′,5′-di-tert-butyl-2′-hydroxyphenyl)-5-chlorobenzotriazole, 2-(3′-tert-butyl-2′-hydroxy-5′-methylphenyl)-5-chlorobenzotriazole, 2-(3′-sec-butyl-5′-tert-butyl-2′-hydroxyphenyl)-benzotriazole, 2-(2′-hydroxy-4′-octyloxyphenyl)-benzotriazole, 2-(3′,5′-di-tert-amyl-2′-hydroxyphenyl)-benzotriazole, 2-(3′,5′-bis(α,α-dimethylbenzyl)-2′-hydroxyphenyl)benzotriazole, a mixture of 2-(3′-tert-butyl-2′-hydroxy-5′-(2-octyloxycarbonylethyl)-phenyl)-5-chlorobenzotriazole, 2-(3′-tert-butyl-5′-[2-(2-ethylhexyloxy)-carbonylethyl]-2′-hydroxyphenyl)-5-chlorobenzotriazole, 2-(3′-tert-butyl-2′-hydroxy-5′-(2-methoxycarbonylethyl)-phenyl)-5-chlorobenzotriazole, 2-(3′-tert-butyl-2′-hydroxy-5′-(2-methoxycarbonylethyl)-phenyl)-benzotriazole, 2-(3′-tert-butyl-2′-hydroxy-5′-(2-octyloxycarbonylethyl)-phenyl)-benzotriazole, 2-(3′-tert-butyl-5′-[2-(2-ethylhexyloxy)-carbonylethyl]-2′-hydroxyphenylybenzotriazole, 2-(3′-dodecyl-2′-hydroxy-5′-methylphenyl)-benzotriazole and 2-(3′-tert-butyl-2′-hydroxy-5′-(2-isooctyloxycarbonylethyl)-phenyl-benzotriazole, 2,2′-methylene-bis-[4-(1,1,3,3-tetramethylbutyl)-6-benzotriazol-2-yl-phenol]; the transesterification product of 2-[3′-tert-butyl-5′-(2-methoxycarbonylethyl)-2′-hydroxyphenyl]-benzotriazole with polyethylene glycol 300; [R—CH₂CH₂—COO(CH₂)₃]₂— wherein R=3′-tert-butyl-4′-hydroxy-5′-2H-benzotriazol-2-yl-phenyl.

2. 2-Hydroxybenzophenones, e.g. a 4-hydroxy, 4-methoxy, 4-octyloxy, 4-decyloxy, 4-dodecyloxy, 4-benzyloxy, 4,2′,4′-trihydroxy or 2′-hydroxy-4,4′-dimethoxy derivative.

3. Esters of unsubstituted or substituted benzoic acids. e.g. 4-tert-butyl-phenyl salicylate, phenyl salicylate, octylphenyl salicylate, dibenzoylresorcinol, bis(4-tert-butylbenzoyl)resorcinol, benzoylresorcinol, 3,5-di-tert-butyl-4-hydroxybenzoic acid 2,4-di-tert-butylphenyl ester, 3,5-di-tert-butyl-4-hydroxybenzoic acid hexadecyl ester, 3,5-di-tert-butyl-4-hydroxybenzoic acid octadecyl ester and 3,5-di-tert-butyl-4-hydroxybenzoic acid 2-methyl-4,6-di-tert-butylphenyl ester.

4. Acrylates, e.g. α-cyano-β,β-diphenylacrylic acid ethyl ester or isooctyl ester, α-methoxycarbonylcinnamic acid methyl ester, α-cyano-β-methyl-p-methoxycinnamic acid methyl ester or butyl ester, α-methoxycarbonyl-p-methoxycinnamic acid methyl ester and N—(β-methoxycarbonyl-β-cyanovinyl)-2-methyl-indoline.

5. Sterically hindered amines, e.g. bis(2,2,6,6-tetramethylpiperidyl)sebacate, bis(2,2,6,6-tetramethylpiperidyl)succinate, bis(1,2,2,6,6-pentamethylpiperidyl)sebacate, n-butyl-3,5-ditert-butyl-4-hydroxybenzyl-malonic acid bis(1,2,2,6,6-pentamethylpiperidyl)ester, the condensation product of 1-hydroxyethyl-2,2,6,6-tetramethyl-4-hydroxypiperidine and succinic acid, the condensation product of N,N′-bis(2,2,6,6-tetramethyl-4-piperidyl)hexamethylenediamine and 4-tert-octylamino-2,6-dichloro-1,3,5-s-triazine, tris(2,2,6,6-tetramethyl-4-piperidyl)nitrilotriacetate, tetrakis(2,2,6,6-tetramethyl-4-piperidyl) 1,2,3,4-butanetetraoate, 1,1′-(1,2-ethanediyl)-bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidyl) 2-n-butyl-2-(2-hydroxy-3,5-di-tert-butylbenzyl)malonate, 3-n-octyl-7,7,9,9-tetramethyl-1,3,8-triazaspiro-[4,5]decane-2,4-dione, bis(1-octyloxy-2,2,6,6-tetramethylpiperidyl)sebacate, bis(1-octyloxy-2,2,6,6-tetramethylpiperidyl)succinate, the condensation product of N,N′-bis(2,2,6,6-tetramethyl-4-piperidyl)hexamethylenediamine and 4-morpholino-2,6-dichloro-1,3,5-triazine, the condensation product of 2-chloro-4,6-di(4-n-butylamino-2,2,6,6-tetramethylpiperidyl)-1,3,5-triazine and 1,2-bis(3-aminopropylamino)ethane, the condensation product of 2-chloro-4,6-di(4-n-butylamino-1,2,2,6,6-pentamethylpiperidyl)-1,3,5-triazine and 1,2-bis(3-aminopropylamino)ethane, 8-acetyl-3-dodecyl-7,7,9,9-tetramethyl-1,3,8-triazaspiro[4,5]decane-2,4-dione, 3-dodecyl-1-(2,2,6,6-tetramethyl-4-piperidyl)-pyrrolidine-2,5-dione, 3-dodecyl-1-(1,2,2,6,6-pentamethyl-4-piperidyl)-pyrrolidine-2,5-dione, 2,4-bis[N-(1-cyclohexyloxy-2,2,6-6-tetramethylpiperidin-4-yl)-n-butyl-amino]-6-(2-hydroxyethyl)amino-1,3,5-triazine and the condensation product of 2,4-bis[1-cyclohexyloxy-2,2,6,6-tetramethylpiperidin-4-yl)butylamino]-6-chloro-s-triazine and N,N′-bis(3-aminopropyl)ethylenediamine.

As it is known to the one skilled in the art, type and concentration of the hindered amine has to be carefully selected, if the process of the current invention is performed using a photolatent acid as photolatent catalysts, in order to prevent the inhibition of the curing process.

6. Oxalic acid diamides, e.g. 4,4′-dioctyloxy-oxanilide, 2,2′-diethoxy-oxanilide, 2,2′-dioctyloxy-5,5′-1-tert-butyl oxanilide, 2,2′-didodecyloxy-5,5′-di-tert-butyl oxanilide, 2-ethoxy-2′-ethyl oxanilide, N,N′-bis(3-dimethylaminopropyl)oxalamide, 2-ethoxy-5-tert-butyl-2′-ethyl oxanilide and a mixture thereof with 2-ethoxy-2′-ethyl-5,4′-di-tert-butyl oxanilide, and mixtures of o- and p-methoxy- and of o- and p-ethoxy-disubstituted oxanilides.

7. 2-(2-Hydroxyphenyl)-1,3,5-triazines, e.g. 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-[2-hydroxy-4-(2-hydroxy-3-butyloxy-propyloxy)-phenyl]-4,6-bis(2,4-dimethyl-phenyl)-1,3,5-triazine, 2-[2-hydroxy-4-(2-hydroxy-3-octyloxy-propyloxy)-phenyl]-4,6-bis(2,4-dimethylphenyl)-1,3,5-triazine and 2-[4-dodecyloxy/tridecyloxy-(2-hydroxypropyl)oxy-2-hydroxyphenyl]-4,6-bis(2,4-dimethylphenyl)-1,3,5-triazine.

8. Phosphites and phosphonites, e.g. triphenyl phosphite, diphenylalkyl phosphites, phenyldialkyl phosphites, tris(nonylphenyl)phosphite, trilauryl phosphite, trioctadecyl phosphite, distearyl-pentaerythritol diphosphite, tris(2,4-di-tert-butylphenyl)phosphite, diisodecylpentaerythritol diphosphite, bis(2,4-di-tert-butylphenyl)pentaerythritol diphosphite, bis(2,6-di-tertbutyl-4-methylphenyl)pentaerythritol diphosphite, bis-isodecyloxy-pentaerythritol diphosphite, bis(2,4-di-tert-butyl-6-methylphenyl)pentaerythritol diphosphite, bis(2,4,6-tri-tert-butylphenyl)pentaerythritol diphosphite, tristearyl sorbitol triphosphite, tetrakis(2,4-di-tert-butylphenyl)-4,4′-biphenylene diphosphonite, 6-isooctyloxy-2,4,8,10-tetra-tert-butyl-12H-dibenzo[d,g]-1,3,2-dioxaphosphocine, 6-fluoro-2,4,8,10-tetra-tert-butyl-12-methyl-dibenzo[d,g]-1,3,2-dioxaphosphocine, bis(2,4-di-tert-butyl-6-methylphenyl)methyl phosphite and bis(2,4-di-tertbutyl-6-methylphenyl)ethyl phosphite.

9. Further inorganic compounds, e.g. nano-titanium dioxide

Examples of UV absorbers and light stabilisers suitable as components (e) also include “Krypto-UVA” as described e.g. in EP 180 548. It is also possible to use latent UV absorbers, as described e.g. by Hida et al in RadTech Asia 97, 1997, page 212.

The proportion of light stabilisers (e) in the formulations is, for example, from 0.01 to 10% by weight, for example from 0.05 to 5% by weight, especially from 0.1 to 5% by weight, based on the binder solid. The concentrations to be used vary according to the layer thickness of the coating. The thinner the layer, the higher must be the concentration of component (e) that is chosen. This will be known to the person skilled in the art and is widely described in the literature.

Other additives customary in the art, e.g. antistatics, flow improvers and adhesion enhancers, can also be used.

It is also possible for chain-transfer reagents customary in the art to be added to the compositions. Examples are mercaptans, amines and benzothiazole.

The curing operation especially of pigmented compositions (pigmented e.g. with titanium dioxide) can be assisted, by the addition as additional additive (h) of a component that forms free radicals under thermal conditions, e.g. an azo compound, such as 2,2′-azobis(4-methoxy-2,4-dimethylvaleronitrile), a triazene, a diazosulfide, pentazadiene or a peroxy compound, for example hydroperoxide or peroxycarbonate, e.g. tert-butyl hydroperoxide, as described e.g. in EP 245 639.

It is also possible to add as additive (h) additives for increasing the mechanical stability, e.g. for increasing scratch-resistance, in the form of nanoparticles. Examples are disclosed in EP114917.

Further customary additives (h)—according to the intended use—are fluorescent whitening agents, fillers, pigments, white and coloured pigments, dyes, antistatics, wetting agents and flow improvers.

For curing thick and pigmented coatings, the addition of glass microspheres or pulverised glass fibers, as described e.g. in U.S. Pat. No. 5,013,768, is suitable.

The choice of additives is governed by the field of use in question and the properties desired for that field. The above-described additives (h) are customary in the art and are accordingly used in the amounts customary in the art.

The proportion of additional additives (h) in the formulations according to the invention is, for example, from 0.01 to 10% by weight, for example from 0.05 to 5% by weight, especially from 0.1 to 5% by weight.

Crosslinking can be accelerated by the addition of photosensitisers (f) which shift or broaden the spectral sensitivity. Such photosensitisers are especially aromatic carbonyl compounds, for example benzophenone derivatives, thioxanthone derivatives, especially isopropylthioxanthone, anthraquinone derivatives and 3-acylcoumarin derivatives, terphenyls, styryl-ketones, as well as 3-(aroylmethylene)-thiazolines, camphorquinone, and also eosin, rhodamine and erythrosine dyes.

The amines mentioned above, for example, can also be considered as photosensitisers.

Further examples of such photosensitisers are

### 1. Thioxanthones

Thioxanthone, 2-isopropylthioxanthone, 3-isopropylthioxanthone, 2-chlorothioxanthone, 2-dodecylthioxanthone, 1-chloro-4-propoxythioxanthone, 2,4-diethylthioxanthone, 2,4-dimethylthioxanthone, 1-methoxycarbonylthioxanthone, 2-ethoxycarbonylthioxanthone, 3-(2-methoxyethoxycarbonyl)-thioxanthone, 4-butoxycarbonylthioxanthone, 3-butoxycarbonyl-7-methylthioxanthone, 1-cyano-3-chlorothioxanthone, 1-ethoxycarbonyl-3-chlorothioxanthone, 1-ethoxycarbonyl-3-ethoxythioxanthone, 1-ethoxycarbonyl-3-aminothioxanthone, 1-ethoxycarbonyl-3-phenylsulfurylthioxanthone, 3,4-di[2-(2-methoxyethoxy)ethoxycarbonyl]thioxanthone, 1-ethoxycarbonyl-3-(1-methyl-1-morpholinoethyl)-thioxanthone, 2-methyl-6-dimethoxymethyl-thioxanthone, 2-methyl-6-(1,1-dimethoxybenzyl)-thioxanthone, 2-morpholinomethylthioxanthone, 2-methyl-6-morpholinomethylthioxanthone, N-allylthioxanthone-3,4-dicarboximide, N-octylthioxanthone-3,4-dicarboximide, N-(1,1,3,3-tetramethylbutyl)-thioxanthone-3,4-dicarboximide, 1-phenoxythioxanthone, 6-ethoxycarbonyl-2-methoxythioxanthone, 6-ethoxycarbonyl-2-methylthioxanthone, thioxanthone-2-polyethylene glycol ester, 2-hydroxy-3-(3,4-dimethyl-9-oxo-9H-thioxanthon-2-yloxy)-N,N,N-trimethyl-1-propanaminium chloride;

### 2. Benzophenones

Benzophenone, 4-phenylbenzophenone, 4-methoxybenzophenone, 4,4′-dimethoxybenzophenone, 4,4′-dimethylbenzophenone, 3-methyl-4′-phenyl-benzophenone, 2,4,6-trimethyl-4′-phenyl-benzophenone, 4,4′-dichlorobenzophenone, 4,4′-dimethylaminobenzophenone, 4,4′-diethylaminobenzophenone, 4-methylbenzophenone, 2,4,6-trimethylbenzophenone, 4-(4-methylthiophenyl)-benzophenone, 3,3′-dimethyl-4-methoxybenzophenone, methyl 2-benzoylbenzoate, 4-(2-hydroxyethylthio)-benzophenone, 4-(4-tolylthio)benzophenone, 4-benzoylN,N,N-trimethylbenzenemethanaminium chloride, 2-hydroxy-3-(4-benzoylphenoxy)-N,N,N-trimethyl-1-propanaminium chloride monohydrate, 4-(13-acryloyl-1,4,7,10,13-pentaoxamidecyl)-benzophenone, 4-benzoyl-N,N-dimethyl-N-[2-(1-oxo-2-propenyl)oxy]ethyl-benzenemethanaminium chloride, 2,4,6-trimethyl-4′-phenyl-benzophenone, 3-methyl-4′-phenyl-benzophenone;

### 3. 3-Acylcoumarins

3-Benzoylcoumarin, 3-benzoyl-7-methoxycoumarin, 3-benzoyl-5,7-di(propoxy)coumarin, 3-benzoyl-6,8-dichlorocoumarin, 3-benzoyl-6-chlorocoumarin, 3,3′-carbonyl-bis[5,7-di(propoxy)coumarin], 3,3′-carbonyl-bis(7-methoxycoumarin), 3,3′-carbonyl-bis(7-diethylaminocoumarin), 3-isobutyroylcoumarin, 3-benzoyl-5,7-dimethoxycoumarin, 3-benzoyl-5,7-diethoxycoumarin, 3-benzoyl-5,7-dibutoxycoumarin, 3-benzoyl-5,7-di(methoxyethoxy)-coumarin, 3-benzoyl-5,7-di(allyloxy)coumarin, 3-benzoyl-7-dimethylaminocoumarin, 3-benzoyl-7-diethylaminocoumarin, 3-isobutyroyl-7-dimethylaminocoumarin, 5,7-dimethoxy-3-(1-naphthoyl)-coumarin, 5,7-dimethoxy-3-(1-naphthoyl)-coumarin, 3-benzoylbenzo[f]coumarin, 7-diethylamino-3-thienoylcoumarin, 3-(4-cyanobenzoyl)-5,7-dimethoxycoumarin;

### 4. 3-(Aroylmethylene)-thiazolines

3-Methyl-2-benzoylmethylene-β-naphthothiazoline, 3-methyl-2-benzoylmethylene-benzothiazoline, 3-ethyl-2-propionylmethylene-β-naphthothiazoline;

### 5. Other Carbonyl Compounds

Acetophenone, 3-methoxyacetophenone, 4-phenylacetophenone, benzil, 2-acetylnaphthalene, 2-naphthaldehyde, 9,10-anthraquinone, 9-fluorenone, dibenzosuberone, xanthone, 2,5-bis(4-diethylaminobenzylidene)cyclopentanone, α-(para-dimethylaminobenzylidene)ketones, such as 2-(4-dimethylamino-benzylidene)-indan-1-one or 3-(4-dimethylamino-phenyl)-1-indan-5-yl-propenone, 3-phenylthiophthalimide, N-methyl-3,5-di(ethylthio)phthalimide.

The proportion of sensitisers (f) in the formulations according to the invention is, for example, from 0.01 to 10% by weight, for example from 0.05 to 5% by weight, especially from 0.1 to 5% by weight.

The formulations may also comprise dyes and/or white or coloured pigments (g). Inorganic or organic pigments may be used. Such additives are known to the person skilled in the art, some examples being titanium dioxide pigments, e.g. of the rutile or anatase type, carbon black, zinc oxide, such as zinc white, iron oxides, such as iron oxide yellow, iron oxide red, chromium yellow, chromium green, nickel titanium yellow, ultramarine blue, cobalt blue, bismuth vanadate, cadmium yellow and cadmium red. Examples of organic pigments are mono- or bis-azo pigments, and also metal complexes thereof, phthalocyanine pigments, polycyclic pigments, for example perylene, anthraquinone, thioindigo, quinacridone or triphenylmethane pigments, and also diketo-pyrrolo-pyrrole, isoindolinone, e.g. tetrachloroisoindolinone, isoindoline, dioxazine, benzimidazolone and quinophthalone pigments.

The pigments can be used in the formulations individually or in admixture.

Depending upon the intended use, the pigments are added to the formulations in the amounts customary in the art, for example in an amount of from 0.1 to 60% by weight, from 0.1 to 30% by weight or from 10 to 30% by weight, based on the total mass.

The formulations may also, for example, comprise organic dyes of an extremely wide variety of classes. Examples are azo dyes, methine dyes, anthraquinone dyes and metal complex dyes. Customary concentrations are, for example, from 0.1 to 20%, especially from 1 to 5%, based on the total mass.

The process according to the present invention is in particular suitable for curing and further processing pigmented formulations. Accordingly, a process wherein the composition of matter comprising the photocatalyst additionally comprises a dye or pigment (g) is preferred. Preferably said formulations are base-catalysed curing.

Depending upon the formulation used, it is also possible to use as stabilisers compounds that neutralise acids, especially amines. Suitable systems are described, for example, in JP-A 11-199610. Examples are pyridine and derivatives thereof, N-alkyl- or N,N-dialkylanilines, pyrazine derivatives, pyrrole derivatives etc.

The invention relates also to a process as described above wherein the composition comprises, in addition to the photolatent component (a), other additives (h), sensitiser compounds (e and/or dyes or pigments (9).

Interesting is also a process as described above wherein the composition comprises as further additive (e) at least one light stabiliser or/and at least one UV absorber compound.

Further, the composition can comprise filler materials, clear ones, but also optically opaque materials. Optically opaque in this connection means opaque to the irradiation that is used to trigger the catalyst. Examples are fabrics, canvas, tissues, fibers, filaments, both of natural or synthetic kind, e.g. polymers, nylon, polyesters etc., reinforced materials, putties, glass, carbon black, etc.

According to the process of the present invention said compositions, comprising a photolatent catalyst are activated by irradiation prior to the further processing and curing.

It is, however, also possible to cure a composition of matter, comprising one or more of the above-described fillers or pigments, dyes or other additives, and not necessarily a photolatent catalyst, by mixing said “filled” and optionally optically opaque composition, i.e. an opaque paste, into a clear composition, that comprises a photolatent catalyst and already has been activated by irradiation prior to the mixing.

In other words, in a process according to the invention, after the irradiation of the composition of matter (optically transparent of the radiation), comprising a photolatent catalyst, another curable composition, for example comprising optically opaque fillers or additives, is mixed in and the resulting mixture cures via the previously activated composition.

It is also possible to mix in other additives or components after the activation of a composition of matter comprising the photolatent catalyst by the irradiation step. Said composition then may be applied to a substrate or be further processed in another way.

Suitable radiation sources, for the irradiation of the composition of matter are radiation sources that emit radiation of a wavelength of approximately from 150 to 1500, for example from 180 to 1000, or preferably from 190 to 700 nanometers, electron-beam (e-beam) radiation and high-energy electromagnetic radiation such as X-rays, as well as microwave radiation. Both, point sources and planiform projectors (lamp carpets) are suitable. Examples are: carbon arc lamps, xenon arc lamps, medium pressure, high pressure and low pressure mercury lamps, optionally doped with metal halides (metal halide lamps), microwave-excited metal vapour lamps, excimer lamps, superactinic fluorescent tubes, fluorescent lamps, argon filament lamps, electronic flash lamps, strobe light, photographic flood lights, cold flat tile-like UV-VIS sources (such as EXFO Photonic solutions), electron beams and X-ray beams generated by means of synchrotrons or laser plasma. The distance between the radiation source and the composition of matter to be irradiated can vary, for example, from 2 cm to 150 cm, according to the intended use and the type and/or strength of the radiation source. Suitable radiation sources are especially mercury vapour lamps, in particular medium and high pressure mercury lamps, from the radiation of which emission lines at other wavelengths can, if desired, be filtered out. That is especially the case for relatively short wavelength radiation. It is, however, also possible to use low energy lamps (for example fluorescent tubes) that are capable of emitting in the appropriate wavelength range, e.g. Philips TLO3 lamps. Another type of radiation source that can be used are the light emitting diodes (LED) that emit at different wavelengths throughout the whole spectrum either as small band emitting source or as broad band (white light) source. Also suitable are laser radiation sources, for example excimer lasers, such as Kr-F lasers for irradiation at 248 nm, Ar—F lasers at 193 nm, or F₂ laser at 157 nm. Lasers in the visible range and in the infrared range can also be used. As a light source further EUV (Extreme Ultra Violet) at 13 nm is also suitable. A suitable laser-beam source is, for example, the argon-ion laser, which emits radiation at wavelengths of 454, 458, 466, 472, 478, 488 and 514 nanometers. Nd-YAG-lasers emitting light at 1064 nm and its second and third harmonic (532 nm and 355 nm respectively) can also be used. Also suitable is, for example, a helium/cadmium laser having an emission at 442 nm or lasers that emit in the UV range. Further, radiation of relatively low intensity is suitable. Such radiation includes, for example, daylight (sunlight), and radiation sources equivalent to daylight. Sunlight differs in spectral composition and intensity from the light of the artificial radiation sources customarily used in UV curing. The absorption characteristics of the compounds according to the invention are as well suitable for exploiting sunlight as a natural source of radiation for curing. Daylight-equivalent artificial light sources that can be used to activate the compounds according to the invention are to be understood as being projectors of low intensity, such as certain fluorescent lamps, for example the Philips TLO5 special fluorescent lamp or the Philips TLO9 special fluorescent lamp.

It is also possible to cure via treatment with a plasma gas. Possible ways of obtaining plasmas under vacuum conditions have been described frequently in the literature.

The irradiation of the composition of matter can for example be effected directly, can, however, also take place behind a transparent layer (e.g. a pane of glass or a sheet of plastics).

The irradiation of the composition of matter, prior to the application of said composition to a substrate, or prior to the further processing of said composition can for example be effected by just irradiating the composition in the container or vessel, where it is prepared or stored.

The irradiation source in this case for example is positioned above the vessel, e.g. at the stirrer. Another possibility is to use a transparent container e.g. from glass or plastic, and irradiate the composition directly through the container. Transparent in the context of this invention means that the radiation used for activation can pass the material. In this case the radiation sources are positioned outside the container. The container can, for example be covered with a lamp carpet on the outside.

Another variant of irradiating the composition of matter is to bring a lamp directly into the formulation, e.g. by protecting the lamp by a transparent container and inserting said container into the container comprising the formulation to be irradiated. It is also possible to incorporate the radiation source in a part of the equipment used for preparing the formulation, e.g. in the stirrer. Furthermore irradiation can be performed using a light guide that immerses into the formulation.

Further, the container with the composition of matter to be irradiated can be placed into a chamber provided with a suitable irradiation source or several irradiation sources, e.g. on the top, on the top and one, on the top and only several or all side panels of the chamber, or even the and all sides, even including the bottom of the chamber in case the container comprising the formulation is transparent.

Irradiation further can be effected shortly before the application of the formulation to a substrate, by for example using a spraying device with a transparent inlet pipe and positioning a suitable irradiation source on top of the transparent part of said pipe. The irradiation source can also be placed after the outlet of the spraying gun in order to irradiate the spray vapour. Further, the irradiation source can be placed in or at the outside of the nozzle of the spraying device.

Suitable irradiation devices that are known to the person skilled in the art are for example immersion well type apparatus as e.g. the Ace reactors offered e.g. by Sigma-Aldrich, merry-go-round photoreactors, annular type irradiation apparatus such as the Rayonette photochemical reactor, flow-trough photochemical reactors (e.g. J. Cooke, G. Austin, M. J. McGarrity, WO 9635508), exposure chambers such as those offered by Luzchem Inc. with top irradiation exposure (e.g. Luzchem LZC-1/LCZ-PAP), side irradiation exposure (e.g. Luzchem LZC-5/LCZ-ORG), or top-side irradiation exposure (e.g. Luzchem LZC-4V); multilamp-type apparatus, tank-type photoreactors, photoreactors with light emitting devices suspended in the reaction mixture (e.g. JP 59059246 A2), steel photochemical reactors (e.g. L. Teodorescu, G. Musca, E. Mocanu, H. Culetu, N. Rada, RO 93292 BI), flow reactors (e.g. D. W. Clark et al Icarus 200, 147, 282), falling film photoreactors (e.g. H. Ehrich et al, Chimia 2002, 56, 647), fountain photoreactors, oscillatory flow photoreactors, draft-tube baffled photoreactor, continuous annular photoreactor, semi-continuous photochemical reactors (e.g. M. Herbert, J. Ollivier, G. Fremy, WO 202081080 A1), tubular photoreactor (e.g. A. Tkac, CS 249894 B1), thin-film cascade photoreactors, high fluence laser photochemical reactors (e.g. L. M. Gantayet, S. Sethi, Adv. Chem. Eng. Nucl. Process Ind. 1994, 146), microfabricated photoreactors H. Lu et al, Lab on a Chip 2001, 1, 22), small scale reactors (James E. Gano, Andrew J. Gano, Patricia Garry, and Padmanabhan Sekher, J. Chem. Edu. 2002, 79(11) 1361), platform reactors.

An overview on available photoreactors is, for example, given by A. M. Braun, M.-T. Maurette, E. Oliveros, “Photochemical Technology”, Wiley, Chchester, West Sussex, England/New York 1993; A. M. Braun, M.-T. Maurette, E. Oliveros, D. F. Ollis, N. Serpone, “Industrial Photochemistry”, M. L. Viriot, J. C. André, A. M. Braun, eds., CPIC-ENSIC, Nancy, 1990, Vol. A, 253-301 “Photochemical reactors”; or in “Chemical Reactor Design and Technology: Overview of the New Developments of Energy and Photochemical Reactor Technologies. Projections for the 90's” by NATO Advanced Study Institute on “Chemical Reactor Design and Technologies, Hugo I. De Lasa (Editor), North Atlantic Treaty Organization Scientific Affairs Division. Fundamentals of photoreactor engineering are reported in J. Costa Lopez, Afinidad 1977, 34(343), 19. A discussion of photochemical reaction engineering fundamentals can also be found in L. Rizzuti, A. Brucato, NATO ASI Series, Series C: Mathematical and Physical Sciences (1988), 237, 623.

An embodiment of the invention also is a process, wherein the irradiation is performed in a flask, a tank, in a pump cycle, in a continuous irradiation device, at the outlet of an evaporator, outside or inside of a spray gun, in conducting tubes or in an ink-jet printing machine.

In particular, the composition of matter is irradiated directly in the storage tank and subsequently subjected to the further processing.

The photochemical activation can be performed either in batch or continuous manner.

Accordingly, another subject of the invention is a process, characterized in that it is conducted continuously by pumping the composition of matter, comprising the photolatent catalyst (a) from a storage tank via an inlet pipe past a radiation source directly to the application means.

The time window between irradiation and curing of the formulation, which allows further processing steps to be applied, can be adjusted between 0.1 seconds and several days, e.g. 7 days, preferred between 1 second and 24 hours, most preferred between 2 seconds and 8 hours.

The time window can be adjusted as required by appropriate selection and combination of the photolatent catalyst, sensitizer, irradiation source and formulation components.

A process, wherein the further processing is carried out 0.5 seconds to 7 days after the irradiation of the composition of matter therefore is also subject of the invention.

Preferably the process according to the invention is conducted as

a process for the coating of substrates, wherein

(A) a composition comprising
   (b) an acid-catalysed curing component and
   (a1) a photolatent acid compound as defined above;
or
(B) a composition comprising
   (c) a base-catalysed curing component and
   (a2) a photolatent base compound as defined above;
or a mixture of (A) and (B)
   is subjected to irradiation with electromagnetic radiation, in particular UV-light, and subsequently applied to a substrate.

Preferably, the composition of matter in the process according to the invention is a laquer formulation, or optionally an adhesive formulation, comprising a polyol in combination with an isocyanate and as photolatent catalyst a photolatent base (a2) of the formula VIII, VIIIa and VIIIb 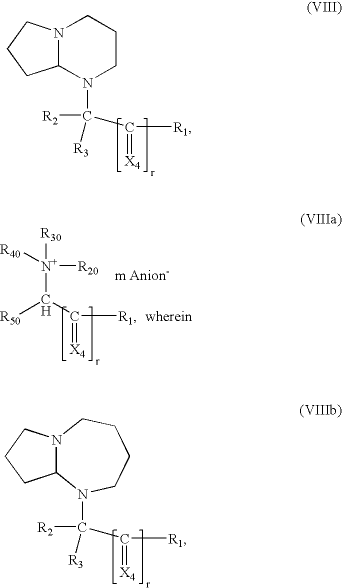
wherein r is 0 or 1;
X₄ is CH₂ or O;
R₂ and R₃ are each independently of the other hydrogen or C₁-C₂₀alkyl;
R₁ is unsubstituted or C₁-C₁₂alkyl- or C₁-C₁₂alkoxy-substituted phenyl, naphthyl or biphenylyl;
R₂₀, R₃₀ and R₄₀, together with the linking nitrogen atom, are a group of the structural formula 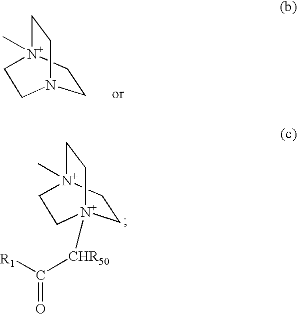
Anion is any anion capable to form the salt; and
m is the number of positively charged N-atoms in the molecule.

Suitable polyols and isocyanates in general are as described above. Preferred suitable polyols and isocyanates for an adhesive formulation are given later on in this context.

Further interesting is a process for the application of a photolatent catalyst (a) wherein a composition of matter, comprising said catalyst, is subjected to irradiation before being further processed, wherein the composition of matter is a laquer formulation, or optionally an adhesive formulation, comprising an epoxide component and as photolatent catalyst a photolatent base (a2) of the formula VIIIa, 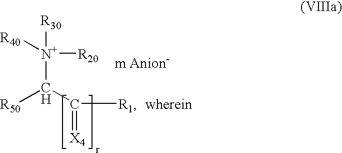
r is 0 or 1;
X₄ is CH₂ or O;
R₁ is unsubstituted or C₁-C₁₂alkyl- or C₁-C₁₂alkoxy-substituted phenyl, naphthyl or biphenylyl;
R₂₀, R₃₀ and R₄₀, together with the linking nitrogen atom, are a group of the structural formula (a), (b) or (c) 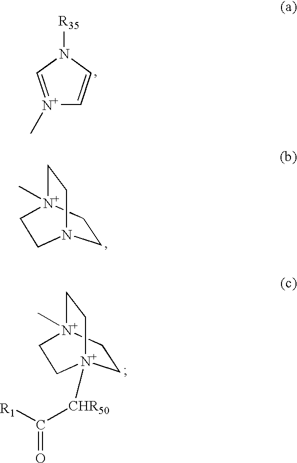
R₃₅ is hydrogen or C₁-C₁₈alkyl;
Anion is any anion capable to form the salt; and
m is the number of positively charged N-atoms in the molecule.

Suitable epoxide components are described above. Preferred are epoxides of the Bisphenol A type.

The composition of matter in the process as described above, concerning laquer and adhesive formulations, preferably is an adhesive.

As already mentioned above the process of the invention is also useful for the adhesive application, e.g. laminating, structure or pressure sensitive adhesives, such as for example pressure sensitive hot-melt adhesives. In particular also in the lamination process of non-transparent substrates, where the activation of the adhesive formulation via irradiation with light can take place prior to the bringing together of the two parts to be laminated.

The adhesive composition typically is an acid-catalysed or base-catalysed curable formulation based on epoxy components or isocyanate components forming polyurethanes. Suitable components of the epoxy and isocyanate type are already described above, as well as corresponding photolatent bases and photolatent acids.

Examples for suitable polyurethane adhesives, also suitable in the presently claimed process are given in US 2005/027091. Said formulations comprise (a) aliphatic or alicyclic polyisocyanate having a functionality ≦3, and (b) a mixture of ≧1 polypropylene glycol diol and polypropylene glycol triol (the ratio of the number of diol OH groups to the number of triol OH groups <10 and the ratio of NCO groups to the number of OH groups 0.95<n<1.05, wherein diols having a molecular weight ≦1000 are applied together with triols having a molecular weight ≧1000 and diols having a molecular weight >1000 are applied together with triols having molecular weight <1000. To said compositions is added a photolatent catalyst as described above in an amount as described above.

More suitable formulations of the polyurethane kind are for example disclosed in US 2004/265529, US 2005/019560, WO 04/055087, U.S. Pat. No. 6,596,787.

Also 1-component polyurethane adhesives as are disclosed in WO 03/050155 can be prepared according to the process of the present application, employing as the amine catalyst a photolatent base as described above.

Accordingly, a process for the application of a photolatent catalyst (a), wherein the composition of matter is an adhesive is also of interest. A process as described above, wherein the photolatent catalyst is a photolatent base (a2) and the composition of matter is a base-catalysed curable compound (c) is also of interest.

The process as described in this invention can also be applied repeatedly. In doing so, a first composition according to this invention is activated by irradiation and subsequently further processed as required. Next, a second composition is activated and further processed in the same application. This process can be repeated as many times as, required. Commonly known as wet in wet application process. The photolatent catalysts used in the different steps may be the same or different and may independently for each step be either a photolatent acid or a photolatent base. Also the base-catalysed or acid-catalysed curable components in the different steps may be the same or differ in each step. An example for such an application is e.g. a multilayer coating, where for example a first coating layer is applied as a primer on the carrier material, followed by a second layer that may contain a pigment and on top a third layer that is a protective clear coat. After all layers are applied the system is cured for example upon application of heat. Since the previous layer is not yet fully cured when the next layer is applied, some mixing of the different coating materials at the interface between the layers can occur, resulting in an improved adhesion of the different layers.

It is not necessary in such multi-step proceedings for each layer to be an activated coating according to the present process. It is also possible to add for example only one activated layer and a further layer without a catalyst. The activated layer can then function as a kind of binder or adhesive as already mentioned before.

In another embodiment of the invention the process is repeatedly applied, the photocatalyst in each repetition step being the same or different from the other steps and independently being either a photolatent acid and/or a photolatent base.

In said repetitive process it is not necessary to undergo a further processing step, e.g. curing, directly after the activation and application of the composition. Each single formulation is activated and applied one after the other and finally the curing for all coatings is effected in one step. It is, however, also possible to effect the curing of each coating directly after the activation and the application before the next activated coating is applied.

“Activated” in the connection of this application means, that the composition of matter, comprising the photolatent catalyst has been subjected to irradiation, which then activates the photolatent catalyst in the formulation.

In the process as described above, one step or more steps may be conducted with compositions comprising a photolatent catalyst (activated prior to the application) and one or more compositions being loaded with highly opaque fillers and/or pigments and only optionally comprising a photolatent catalyst. That means a first composition of matter comprising a photolatent catalyst is activated by irradiation and then applied to a substrate, a second composition of matter, being the same or different from the first one and optionally comprising a photolatent catalyst, is applied on top of the first one etc. After the application of all wanted coatings the curing is effected, for example by heat and/or further irradiation.

The process of the invention is for example repeated from two to ten times, e.g. two to 5 times or two to 3 times. Said composition highly loaded with opaque fillers and/or pigments can be used in the form of a past, i.e. an opaque paste.

A subject of the invention also is a process, wherein the further processing is an additional curing step using UV-light and/or heat.

Another embodiment of the invention is a substrate covered with a composition of matter according to the process of claim 1.

The compositions, which are subject of the process according to the invention can be used for various purposes, in the preparation of surface coatings, printing inks, e.g. screen printing inks, inks for offset- or flexo printing, as a clear finish, as a white or colored finish, for example for wood or metal, as powder coating, as a coating material, inter alia for paper, wood, metal or plastic, as a daylight-curable coating for the marking of buildings and roadmarking, as dental filling compositions, as adhesives, as pressure-sensitive adhesives, as laminating resins, for producing three-dimensional articles by mass curing, or by injection molding, to produce composite materials (for example styrenic polyesters, which may, if desired, contain glass fibres and/or other fibres and other auxiliaries) and other thick-layered compositions, for coating or sealing electronic components and integrated circuits, or as coatings for optical fibres, or for producing optical lenses, e.g. contact lenses or Fresnel lenses. The compositions in the process according to the invention are further suitable for the production of medical equipment, auxiliaries or implants and for the preparation of gels with thermotropic properties, as for example described in DE 19700064 and EP 678534.

The compositions cured by the process according to the invention can, for example, also be used as repair materials and as putty materials.

Structured devices can be produced when the processing step includes a printing or stamping step where the activated formulation is image-wise applied on a suitable supporting material. This is for example possible by using the soft lithography technique developed by G. Whitesides (e.g. Xia, Y., and G. M. Whitesides, Extending microcontact printing as a microlithographic technique. Langmuir 1997, 13, 2059-67; Xia, Y., D. Qin, and G. M. Whitesides, Microcontact printing with a cylindrical rolling stamp: A practical step toward automatic manufacturing of patterns with submicrometer-sized features. Adv. Mater. 1996, 8, 1015-17. The formulation thus applied is then crosslinked by the acid or base-catalysed curing process. The microstructures thus obtained can e.g. be used for reproduction techniques, for image recording techniques, to produce printing plates, as etch resists, solder resists, electroplating resists, or permanent resists, both liquid and dry films, as TFT resists, for printed circuit boards and electronic circuits, as resists to manufacture color filters, e.g. for generating red, green and blue color pixels and a black matrix; for a variety of display applications or to generate structures in the manufacturing process of plasma-display panels and electroluminescence displays.

The process of the invention can also be used to prepare powder coatings.

By “powder coating compositions” or “powder coatings” is meant the definition as described in “Ullmann's Encyclopedia of Industrial Chemistry, 5th, Completely Revised Edition, Vol. A 18”, pages 438 to 444 (1991) in Section 3.4. By powder coatings are meant thermoplastic or bakable, crosslinkable polymers, which are applied in powder form to predominantly metallic substrates. The way in which the powder is brought into contact with the workpiece that is to be coated typifies the various application techniques, such as electrostatic powder spraying, electrostatic fluidized-bed sintering, fixed bed sintering, fluidized-bed sintering, rotational sintering or centrifugal sintering.

According to the invention the powder coating formulation is activated prior to the application. Preferred organic film-forming binders for the powder coating compositions of the invention are stoving systems based, for example, on epoxy resins, polyester-hydroxyalkylamides, polyester-glycolurils, epoxy-polyester resins, polyester-triglycidyl isocyanurates, hydroxy-functional polyester-blocked polyisocyanates, hydroxy-functional polyester-uretidiones, acrylate resins with hardener, or mixtures of such resins.

Polyesters are in general hydroxy- or carboxy-functional and are normally prepared by condensation of diols and dicarboxylic acids. By adding polyols and/or polyacids, branched polyesters are obtained which then give rise, in the course of baking in the presence of crosslinkers, to network structures which give the coating the desired physical properties, such as scratch resistance, impact strength and flexural strength. Instead of multifunctional acids it is also possible to use anhydrides or acid chlorides, such as maleic anhydride, itaconic anhydride, phthalic anhydride, terephthalic anhydride, hexahydroterephthalic anhydride, trimellitic anhydride, pyromellitic dianhydride, succinic anhydride, etc. It is also possible to use simple esters such as dimethyl terephthalate for example, in which case the polymerization proceeds by transesterification with elimination of the volatile alcohol. Likewise practicable is a preparation by a combination of transesterification and condensation. Polyesters can be prepared, furthermore, by polycondensation of hydroxycarboxylic acids such as 12-hydroxystearic acid and hydroxypivalic acid, or of the corresponding lactones, such as ε-caprolactone, for example. Examples of dicarboxylic acids and polyacids include terephthalic, isophthalic, adipic, azelaic, sebacic, 1,12-dodecanedioic, pyromellitic, 3,6-dichlorophthalic, succinic, 1,3-cyclohexanedicarboxylic and 1,4-cyclohexanedicarboxylic acids. Examples of diols and polyols include ethylene glycol, propylene glycol, glycerol, hexanetriol, hexane-2,5-diol, hexane-1,6-diol, pentaerythritol, sorbitol, neopentyl glycol, trimethylolethane, trimethyllolpropane, tris-1,4-cyclohexanedimethanol, trimethylpentanediol, 2,2-diethyl-1,3-propanediol, 2-methyl-2-butyl-1,3-propanediol, esterdiol 204 (ester of hydroxypivalic acid and neopentyl glycol), hydrogenated bisphenol A, bisphenol A, hydroxypivalic acid, hydroxypivalate esters, 2-butyl-2-ethyl-1,3-propanediol, 1,4-butanediol, 2-butene-1,4-diol, 2-butyne-1,4-diol or 2-methyl-1,3-propanediol.

Suitable crosslinking agents for carboxy-functional polyesters are epoxy compounds such as Novolace-epoxy resins, diglycidyl ethers of bisphenol A, hydrogenated bisphenol A and bisphenol A modified by reaction with, for example, aliphatic dicarboxylic acids. Also suitable are reactive epoxy compounds, such as triglycidyltriazolidine-3,5-dione, the glycidyl esters of polyacids, such as diglycidyl terephthalate and diglycidyl hexahydroterephthalate, hydantoin epoxides (U.S. Pat. No. 4,402,983) and, especially, triglycidyl isocyanurate, epoxidized unsaturated fatty acid esters (for example Uranox® from DSM) and Araldit® PT 910 (Ciba Spezialitätenchemie AG). Further crosslinking agents for carboxy-functional polyesters are β-hydroxyalkylamides (U.S. Pat. No. 4,076,917), such as the predominantly tetrafunctional β-hydroxyalkylamide derivative of adipic acid (Primid® XL552 from Rohm & Haas), for example. Derivatives of melamine, benzoguanimine and glycoluril that have been alkylated with low molecular mass alcohols have also proved suitable. Examples are tetramethylmethoxyglycoluril (Powderlink® 1174 from American Cyanamid). In addition, bis- and trisoxazolidines, such as 1,4-bisoxazolidinobenzene, for example, are also known as crosslinking agents.

More recent are carboxy-functional polyester, which contain chemically bonded epoxy groups and are thus able to crosslink with themselves (Molhoek et al., 22nd Fatipec Congress, 15-19.5.95, Budapest, Vol. 1, 119-132).

In all systems in which an epoxy group or a glycidyl radical reacts with a carboxyl group or with an anhydride in a crosslinking reaction, it is possible to employ catalysts. Examples are amines or metal compounds such as aluminium acetylacetonate or tin octoate, for example.

The polyisocyanate crosslinkers are of particular importance as crosslinking agents for hydroxy-functional polyesters. In order to prevent premature crosslinking, because of the high reactivity of isocyanates, and to obtain good levelling of the melted powder, the polyisocyanates are blocked (internally in the form of a uretidione, or as an adduct with a blocking agent). Blocking agents most commonly employed are ε-caprolactam, methyl ethyl ketoxime or butanone oxime. Other suitable blocking agents for isocyanates are described in the publications by G. B. Guise, G. N. Freeland and G. C. Smith, J. Applied Polymer Science, 23, 353 (1979) and by M. Bock and H.-U. Maier-Westhues in “Progress in Product Development for Powder Coating Technology, XIX th Int. Conf. on Organic Coatings, Science and Technol., Athens, 12-16 July”, 1993. Examples of blocked and unblocked polyisocyanates include 2-methylpentane 1,5-diisocyanate, 2-ethylbutane 1,4-diisocyanate, 3(4)-isocyanatomethyl-1-methylcyclohexyl isocyanate, 3-isocyanatomethyl-3,5,5-trimethylcyclohexane diisocyanate, tris(isocyanatomethyl)benzene, 4,4′-diisocyanatodicyclohexylmethane, 1,4-bis(isocyanatomethyl)cyclohexane, m-tetramethylxylene diisocyanate, p-tetramethylxylene diisocyanate and, in particular, isophorone diisocyanate. For deblocking it is common to add a metallic catalyst, such as tin octoate, dibutyltin oxide or dibutyltin dilaurate, for example, to the polyisocyanate formulation.

Further suitable crosslinking agents for hydroxy-functional polyesters are anhydrides such as trimellitic anhydride and its reaction products with diols and diamines. Further examples of such crosslinking agents are described by T. A. Misev in “Powder Coatings: Chemistry and Technology”, published by J. Wiley & Sons, Chichester on pages 123 and 124.

Polyacrylates, which commonly possess hydroxyl, carboxyl or glycidyl functionality, are also employed as binders for powder coatings. They are prepared by the customary methods, principally from monomers such as styrene and linear or branched C₁-C₈alkyl esters of acrylic or methacrylic acid. In addition, other ethylenically unsaturated compounds, such as divinylbenzene, acrylamide, methacrylamide, butoxymethylacrylamide, acrylonitrile, butadiene, etc., can be added and copolymerized. Hydroxyl functionality is ensured by the copolymerization of hydroxy-functional monomers such as hydroxyethyl acrylate, hydroxyethyl methacrylate, hydroxypropyl acrylate, hydroxypropyl methacrylate, for example. For carboxyl functionality use is made of ethylenically unsaturated acids and anhydrides, such as acrylic, methacrylic, itaconic and crotonic acid, and maleic, itaconic, acrylic or methacrylic anhydrides (U.S. Pat. No. 3,836,604). Glycidyl functionality is provided, as taught in EP-A-0 256 369 and U.S. Pat. No. 3,876,578, by the copolymerization of monomers such as glycidyl acrylate and glycidyl methacrylate. As crosslinking agents for polyacrylates with hydroxyl or carboxyl functionality it is possible in principle to use the same compounds as already described for the polyesters with hydroxyl or carboxyl functionality. Further suitable crosslinking agents are the epoxy compounds of U.S. Pat. No. 0,045,040. Suitable crosslinking agents for polyacrylates with glycidyl functionality are dicarboxylic acids, such as sebacic acid and 1,12-dodecanedicarboxylic acid, and anhydrides, such as bistrimellitic anhydride, for example, and the compounds described in U.S. Pat. No. 3,880,946. DE-A-3 310 545, furthermore, discloses self-crosslinking polyacrylates.

Epoxy resins for powder coatings are usually either Novolaco-epoxy resins or, in particular, those based on aromatic polyols, especially those based on bisphenols such as bisphenol A. Also known are modified bisphenol epoxy resins, from JP-A-58 187 464 (1982). The epoxy resins are employed in combination with crosslinkers from the classes of the solid aliphatic amines, solid aromatic amines, amine adducts, phenolic resins, polyacids and the already described carboxy-functional polyesters. Hardeners deserving of very special mention are the dicyandiamides, which are frequently employed together with a catalyst, examples of which are Lewis acids, boron trifluoride-amine complexes, metal complexes, tertiary or quaternary amines, and imidazoline derivatives, such as 2-methylimidazoline.

Photocuring is of great importance for printings, since the drying time of the ink is a critical factor for the production rate of graphic products, and should be in the order of fractions of seconds. UV-curable inks are particularly important for screen printing and offset inks.

Such printing inks are known to the person skilled in the art, are used widely in the art and are described in the literature. They are, for example, pigmented printing inks and printing inks coloured with dyes.

A printing ink is, for example, a liquid or paste-form dispersion that comprises colorants (pigments or dyes), binders and also optionally solvents and/or optionally water and additives. In a liquid printing ink, the binder and, if applicable, the additives are generally dissolved in a solvent. Customary viscosities in the Brookfield viscometer are, for example, from 20 to 5000 mPa·s, for example from 20 to 1000 mPa·s, for liquid printing inks. For paste-form printing inks, the values range, for example, from 1 to 100 Pa·s, preferably from 5 to 50 Pa·s. The person skilled in the art will be familiar with the ingredients and compositions of printing inks. Suitable pigments, like the printing ink formulations customary in the art, are generally known and widely described.

The printing inks can be used, for example, for intaglio printing, flexographic printing, screen printing, offset printing, lithography or continuous or dropwise ink-jet printing on material pretreated in accordance with the process of the invention using generally known formulations, for example in publishing, packaging or shipping, in logistics, in advertising, in security printing or in the field of office equipment.

Suitable printing inks are both solvent-based printing inks and water-based printing inks. Of interest are, for example, printing inks based on aqueous acrylate.

For intaglio or flexographic printing, a printing ink is usually prepared by dilution of a printing ink concentrate and can then be used in accordance with methods known per se.

The printing inks may, for example, also comprise alkyd systems that dry oxidatively.

The ink usually comprises a pigment or a dye or a combination of pigments or dyes, a dispersant and a binder. It will be understood that the printing inks may comprise further auxiliaries, such as are customary, for example preservatives, anti-oxidants, degassers/defoamers, viscosity regulators, thickeners, flow improvers, anti-settling agents, gloss improvers, lubricants, adhesion promoters, anti-skin agents, matting agents, emulsifiers, stabilisers, hydrophobic agents, light stabilisers, handle improvers, anti-statics, buffer substances, surfactants, humectants and substances that inhibit the growth of fungi and/or bacteria.

The printing inks may also be prepared in customary manner by mixing the individual components together, for example in the desired amount of water.

The printing inks are also suitable, for example, for use in recording systems of the kind in which a printing ink is expressed from a small opening in the form of droplets which are directed towards a substrate on which an image is formed. Suitable substrates are, for example, textile fibre materials, paper, plastics or aluminium foils pretreated by the process according to the invention. Suitable recording systems are e.g. commercially available ink-jet printers.

In particular the printing as the photolatent catalyst comprises

(a1) a photolatent acid, e.g. a triarylsulfonium salt, or an aromatic sulfonium salt of the formula VI as described above;

or wherein the photolatent acid (a1) is a compound selected from the group consisting of aromatic phosphonium salts, aromatic iodonium salts or oxime-based photolatent acids; or

(a2) a photolatent base compound.

Another field where the photocuring process according to the invention can be employed is the coating of metals, in the case, for example, of the coating of metal plates and tubes, cans or bottle caps, cars and other vehicles, e.g. trains, bicycles, airplanes, boats, ships etc., and the photocuring of polymer coatings, for example of floor or wall coverings based on PVC.

Examples of the photocuring of paper coatings are the colourless varnishing of labels, record sleeves and book covers.

Also of interest is the use of the novel process for curing shaped articles made from composite compositions. The composite compound consists of a self-supporting matrix material, for example a glass fibre fabric, or alternatively, for example, plant fibres [cf. K.-P. Mieck, T. Reussmann in Kunststoffe 85 (1995), 366-370], which is impregnated with the activated, i.e. irradiated photocuring formulation. Shaped parts comprising composite compounds attain a high level of mechanical stability and resistance. The novel process can also be employed for the preparation of mouldings, or for impregnating and coating compositions as are described, for example, in EP 7086. Examples of such compositions are gel coat resins, which are subject to stringent requirements regarding curing activity and yellowing resistance, and fibre-reinforced mouldings, for example, light diffusing panels which are planar or have lengthwise or crosswise corrugation. Techniques for producing such mouldings, such as hand lay-up, spray lay-up, centrifugal casting or filament winding, are described, for example, by P. H. Selden in “Glasfaserverstärkte Kunststoffe”, page 610, Springer Verlag Berlin-Heidelberg-New York 1967. Examples of articles which can be produced by these techniques are boats, fibre board or chipboard panels with a double-sided coating of glass fibre-reinforced plastic, pipes, containers, surfboards etc. Further examples of moulding, impregnating and coating compositions are UP resin gel coats for mouldings containing glass fibres (GRP), such as corrugated sheets and paper laminates. Paper laminates may be based on urea resins or melamine resins. Prior to production of the laminate, the gel coat is irradiated and produced on a support (for example a film). The novel process can also be used for producing casting resins or for embedding articles, for example electronic components, etc.

The novel process is suitable, for example, for coating substrates of all kinds, for example wood, textiles, paper, ceramics, glass, plastics such as polyesters, polyethylene terephthalate, polyolefins or cellulose acetate, especially in the form of films, and also metals such as Al, Cu, Ni, Fe, Zn, Mg or Co and GaAs, Si or SiO₂ to which it is for example intended to apply a protective layer.

Coating of the substrates can be carried out by applying to the substrate a liquid composition, a solution or a suspension, which prior to the application has been irradiated. The choice of solvents and the concentration depend principally on the type of composition and on the coating technique. The solvent should be inert, i.e. it should not undergo a chemical reaction with the components and should be able to be removed again, after coating, in the course of drying. Examples of suitable solvents are ketones, ethers and esters, such as methyl ethyl ketone, isobutyl methyl ketone, cyclopentanone, cyclohexanone, N-methylpyrrolidone, dioxane, tetrahydrofuran, 2-methoxyethanol, 2-ethoxyethanol, 1-methoxy-2-propanol, 1,2-dimethoxyethane, ethyl acetate, n-butyl acetate, ethyl 3-ethoxypropionate, 2-methoxypropylacetate, methyl-3-methoxypropionate, 2-heptanone, 2-pentanone, and ethyl lactate.

The solution is applied uniformly to a substrate by means of known coating techniques, for example by spin coating, dip coating, knife coating, curtain coating, brushing, spraying, especially by electrostatic spraying, and reverse-roll coating, and also by means of electrophoretic deposition. It is also possible to apply the photosensitive layer to a temporary, flexible support and then to coat the final substrate, for example a copper-clad circuit board, or a glass substrate by transferring the layer via lamination.

The quantity applied (coat thickness) and the nature of the substrate (layer support) are dependent on the desired field of application. The range of coat thicknesses generally comprises values from about 0.1 μm to more than 100 μm, for example 0.1 μm to 1 cm, preferably 0.5 μm to 1000 μm.

The novel process may additionally be employed for emulsion polymerizations, pearl polymerizations or suspension polymerizations.

Process as described above, wherein the further processing involves

the application of the irradiated composition of matter to a substrate, optionally followed by further mechanical processing steps of the coated substrate, such as bending, cutting, polishing; the preparation of a foam; the preparation of a polymer; the preparation of a fiber; the preparation of a gelcoat; the preparation of a composite, the preparation of an adhesive, the preparation of a clear coating or a pigmented coating, a printing ink, an inkjet ink, or the preparation of a coating that has an additional material incorporated, e.g. sand for sanding paper.

In the process according to the present invention, the further processing may also reside in the preparation of a foam (flexible, rigid, integral or a microcellular foam).

The composition of matter to prepare said foams comprises polyether polyol, polyester polyol or polyurethane compositions

The polyurethanes are obtained, for example, by reacting polyethers, polyesters and polybutadienes which contain terminal hydroxyl groups with aliphatic or aromatic polyisocyanates.

Polyethers and polyesters having terminal hydroxyl groups are known and are prepared, for example, by polymerizing epoxides such as ethylene oxide, propylene oxide, butylene oxide, tetrahydrofuran, styrene oxide or epichlorohydrin with themselves, for example in the presence of BF₃, or by addition reaction of these epoxides, alone or as a mixture or in successsion, with starting components containing reactive hydrogen atoms, such as water, alcohols, ammonia or amines, for example ethylene glycol, propylene 1,3- and 1,2-glycol, trimethylolpropane, 4,4′-dihydroxydiphenylpropane, aniline, ethanolamine or ethylenediamine. Sucrose polyethers are also suitable in accordance with the invention. In many cases preference is given to those polyethers which predominantly (up to 90% by weight, based on all the OH groups present in the polyether) contain primary OH groups. Furthermore, polyethers modified by vinyl polymers, as are formed, for example, by polymerizing styrene and acrylonitrile in the presence of polyethers, are suitable, as are polybutadienes containing OH groups. These compounds generally have molecular weights of 40 and are polyhydroxy compounds, especially compounds containing from two to eight hydroxyl groups, especially those of molecular weight from 800 to 10 000, preferably from 1000 to 6000, for example polyethers containing at least 2, generally 2 to 8, but preferably 2 to 4, hydroxyl groups, as are known for the preparation of homogeneous polyurethanes and cellular polyurethanes.

It is of course possible to employ mixtures of the above compounds containing at least two isocyanate-reactive hydrogen atoms, in particular with a molecular weight of 400-10 000.

Suitable polyisocyanates are aliphatic, cycloaliphatic, araliphatic, aromatic and heterocyclic polyisocyanates, for example ethylene diisocyanate, 1,4-tetramethylene diisocyanate, 1,6-hexamethylene diisocyanate, 1,12-dodecane diisocyanate, cyclobutane 1,3-diisocyanate, cyclohexane 1,3- and -1,4-diisocyanate and also any desired mixtures of these isomers, 1-isocyanato-3,3,5-trimethyl-5-isocyanatomethylcyclohexane, 2,4- and 2,6-hexahydrotolylene diisocyanate and also any desired mixtures of these isomers, hexahydro-1,3- and/or -1,4-phenylene diisocyanate, perhydro-2,4′- and/or -4,4′-diphenylmethanediisocyanate, 1,3- and 1,4-phenylene diisocyanate, 2,4- and 2,6-tolylene diisocyanate, and also any desired mixtures of these isomers, diphenylmethane 2,4′- and/or 4,4′-diisocyanate, naphthylene 1,5-diisocyanate, triphenylmethane 4,4′,4″-triisocyanate, polyphenyl-polymethylene polyisocyanates as are obtained by aniline-formaldehyde condensation followed by phosgenization, m- and p-isocyanatophenylsulfonyl isocyanates, perchlorinated aryl polyisocyanates, polyisocyanates containing carbodiimide groups, polyisocyanates containing allophanate groups, polyisocyanates containing isocyanurate groups, polyisocyanates containing urethane groups, polyisocyanates containing acylated urea groups, polyisocyanates containing biuret groups, polyisocyanates containing ester groups, reaction products of the above-mentioned isocyanates with acetals, and polyisocyanates containing polymeric fatty acid radicals.

It is also possible to employ the isocyanate group-containing distillation residues as they are or dissolved in one or more of the abovementioned polyisocyanates, which are obtained in the course of the industrial preparation of isocyanates. It is additionally possible to use any desired mixtures of the abovementioned polyisocyanates.

Particular preference is given in general to the polyisocyanates which are readily obtainable industrially, for example 2,4- and 2,6-tolylene diisocyanate and any desired mixtures of these isomers (“TDI”), polyphenyl-polymethylene-polyisocyanates as prepared by aniline-formaldehyde condensation followed by phosgenization (“crude MDI”), and polyisocyanates containing carbodiimide, urethane, allophanate, isocyanurate, urea or biuret groups (“modified polyisocyanates”).

Polyurethane foams are preferably prepared from liquid starting components, either the starting materials to be reacted with one another being mixed together in a one-shot process, or a preadduct containing NCO groups that are formed from a polyol and an excess of polyisocyanate being prepared first and then foamed, typically by reaction with water.

According to the process of the present invention the foam composition is irradiated prior to the addition of the isocyanate component.

In the preparation of foams, the foaming is often carried out in moulds. In that case, the reaction mixture is placed in a mould, e.g. after the irradiation and addition of the water component. Suitable mould materials are metals, typically aluminium, or plastics, typically epoxy resins. In the mould, the foamable reaction mixture foams up and forms the moulded article. The foam moulding can be carried out such that the moulding has a cellular surface structure or, alternatively, such that the moulding has a dense skin and a cellular core. In this connection, it is possible to place into the mould a sufficient amount of foamable reaction mixture for the foam obtained to fill the mould exactly. It is, however, also possible to place more foamable reaction mixture into the mould than is required to fill the interior of the mould with foam. In the latter case, therefore, the operation is carried out with overcharging.

In the case of foam moulding, known external release agents, typically silicone oils, are often used concomitantly. It is, however, also possible to use so-called internal release agents, optionally in admixture with external release agents. It is also possible to use cold-curing foams. The foams can, of course, alternatively be prepared by block foaming or by the known double conveyor belt process. These processes can be used to prepare flexible, semi-flexible or hard polyurethane foams.

The foams find the utilities known for such products, for example as mattresses and upholstery in the furniture and automobile industries, as well as for the manufacture of fittings, such as are used in the automobile industry, and finally as sound-insulating compositions and as compositions for heat-insulation and low-temperature insulation, for example in the construction sector or in the refrigeration industry, or in the textile industry, for example as shoulder pads. Said foams are of special interest for example in the automotive industry to prepare for example, arm rests, head restraints, acoustic foam carpets, seats from flexible foams; or e.g. roof liners, hat racks, door panels, arm rests, instrument panels, head-impacts, side-impacts from semi-rigid foams; or use the foam to fill cavities (rigid foams); or to prepare for example steering wheels, air filters, gearshift knobs, spoilers, cable sheetings, head restraints from flexible integral foams.

Accordingly a further subject of the invention is a process for the application of a photolatent catalyst (a), wherein a composition of matter, comprising said catalyst, is subjected to irradiation before being further processed, characterized in that the further processing resides in the preparation of a foam and the composition of matter comprises polyol and isocyante components and as photolatent catalyst a photolatent base (a2).

The following Examples illustrate the invention further but are considered nonlimiting to the scope of the patent claims. As in the remainder of the description and in the patent claims, parts or percentages relate to weight unless otherwise indicated. Where reference is made to alkyl or alkoxy radicals having more than three carbon atoms without any indication of their isomeric form, the respective n-isomers are intended.

### EXAMPLE 1

The following formulations are prepared by mixing the respective ingredients:

Component A

54.78 parts of a hydroxyfunctional poylacrylate (Desmophen A VP LS 2350, provided by Bayer AG)
0.70 parts of a leveling agent Byk 333 (10% in butylacetate, polyether-modified polydimethyl-siloxane, provided by Byk Chemie)
0.50 parts of a leveling agent Byk 355 (solution of polyacrylates, provided by Byk Chemie)
0.55 parts of a defoamer Byk 141 (polysiloxane, provided by Byk Chemie)
18.47 parts of a mixture of xylene/methoxypropylacetate/butylacetate in the ratio 1:1:1
   Component B
20.08 parts of an aliphatic polyisocyanate (Desmodur N 3390, provided by Bayer AG)
   2.8% of the photolatent base 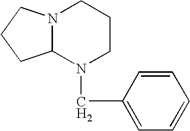
   (PLB-1) are well dissolved in Component A. Prior to the application Component B is added to the samples and homogenised. The thus prepared coating formulation is then placed in a petri dish and irradiated on UV-curing equipment with 2×80 W/cm Hg-bulbs at a line speed of 3 m/min.

The reactivity of the formulations containing is determined with a drying recorder from Byk-Gardner. Following the irradiation of the formulation in the petri dish, the coating is applied on a glass plate, which has a length of 30 cm, with a 75 μm slit coater. The coated glass plate is then positioned on the recorder, where a needle is moving with a constant speed over the coated substrate in the dark. It takes 24 h until the needle passes the whole length of the substrate. After this time the curing phases are determined. Relevant is the time to obtain a tack-free the coating (=phase 3).

A second sample of the formulation comprising Components A and B is subjected to the recorder without having been pre-irradiated. Further, samples without a photolatent catalyst are subjected to the procedure as described above, i.e. with a pre-irradiation step and without.

The results are collected in the following table 1.

TABLE 1
Curing time to achieve a tack-free coating

| | | Pre-irradiation, |
|---|---|---|
| | Curing in the dark, | followed by |
| Catalyst | without pre-irradiation | curing in the dark |
| none | 8 hours | 8 hours |
| photolatent base | 8 hours | 5 hours |
| (PLB-1) | | |

### EXAMPLE 2

### Preparation of Polyether/Polyurethane Soft Foams

0.08 g (0.05% based on the polyol) of an aminic catalyst, 1,5-Diazabicyclo[4.3.0]non-5-en (DBN)

0.24 g (0.15% based on the polyol) of the photolatent catalyst PLB-1 is dissolved in 160 g of a polyether polyol [Lupranol 2082® hydroxyl number 48 mg KOH/g, water content less than 0.1%, acid number less than 0.1 mg KOH/g)]. This sample is then exposed for 2 minutes under a Panacol UVA Lamp UV F 450 W using a blue light filter.

9.6 g of a solution consisting of 1.92 g of a polysiloxane polyoxaalkylene block copolymer foam stabilizer (Tegostab® BF 2370; supplied by Goldschmidt, Germany) and 7.68 g of deionized water are added and the reaction mixture is stirred vigorously for 10 seconds at 2600 rpm. 0.32 g of stannous octoate (Kosmos® 29; supplied by Goldschmidt, Germany) is then added and the reaction mixture is again stirred vigorously for 18 seconds at 2600 rpm. 94.54 g of an isocyanate (Lupranat® T80, supplied by BASF; toluoylene-2,4- and toluoylene-2,6-diisocyanate mixture) is then added with continuous stirring for 5 to 7 seconds at 2600 rpm. The mixture is then poured into a 20×20×20 cm cake-box and in an exothermic reaction a foam block is obtained.

In a second test 0.08 g (0.05% based on the polyol) of a photolatent catalyst (PLB-1) is added to the formulation. The preparation of the foam is performed as described above. Also in this case a foam block is obtained.

### EXAMPLE 3

### Preparation of a Solventless 2P Polyurethane Adhesive Formulation

Component A is prepared by mixing the ingredients indicated below:
50.0% by weight of trifunctional polypropylene polyether polyol (Desmophen® 1380 BT, provided by Bayer)
20.0% by weight of linear polypropylene polyether polyol (Desmophen® 1112 BD, provided by Bayer)
0.21% by weight of a sensitizer, isopropylthioxanthone (Darocure® ITX, provided by Ciba Specialty Chemicals)
2.1% by weight of the photolatent base PLB-1
   Component B:

Aromatic polyisocyanate prepolymer based on diphenylmethane diisocyanate (MDI), Desmodur® E21, provided by Bayer.

(The components A and B are mixed in a ratio A:B=1:0.74.)

### EXAMPLE 3.1

0.5 g of Compound A are placed in an open clear glass crystallizer (layer thickness approx. 1 mm) and irradiated with UV-fluorescent lamps TL 05 (Philips) for 10 min. During irradiation time the liquid is constantly stirred using a magnetic stirring device. After irradiation 0.37 g of Compound B are added and stirred in quickly using a stainless steel spatula. The homogenous formulation is applied on a KBr crystal used for FTIR-Spectroscopy using a wire bar (WFT 34 μm). Immediately after application the crosslinking reaction of isocyanate with polyol is followed by FTIR-spectroscopy using a Perkin Elmer 1600 Spectrometer.

Spectra are measured after 5; 15; 30; 60 and 120 min, setting first spectra a time zero. The decrease of the isocyanate peak height at 2271 cm⁻¹ over time is used as a measure for reactivity of the system. The results are collected in the following table 2, showing the NCO-conversion calculated from the FTIR-measurements.

### EXAMPLE 3.2

0.28 g of Compound A and 0.21 g of Compound B are placed in an open clear glass crystallizer (layer thickness approx. 1 mm) and irradiated with UV-fluorescent lamps TL 05 (Philips) for 10 min. During irradiation time the liquid is constantly stirred using a magnetic stirring device.

After irradiation the formulation is applied on a KBr crystal used for FTIR-Spectroscopy using a wire bar (WFT 34 μm). Immediately after application the reaction is followed using FTIR-spectroscopy as described above. The results are collected in table 2.

TABLE 2

NCO-conversion [%]

| Time t [min] | Example 3.1 | Example 3.2 |
|---|---|---|
| 0 | 0 | 0 |
| 5 | 12.91 | 4.47 |
| 15 | 32.08 | 15.48 |
| 30 | 46.45 | 30.03 |
| 60 | 64.77 | 47.03 |
| 120 | 77.61 | 64.55 |

The remaining film on the crystal after the FTIR-measurements is dry to the touch.

### EXAMPLE 4

### Preparation of a Solventless 2P Polyurethane Adhesive Formulation

0.5 g of Compound A (as described in example 3) are placed in an open clear glass crystallizer (layer thickness approx. 1 mm) and irradiated with UV-fluorescent lamps TL 05 (Philips) for 10 min. During irradiation time the liquid is constantly stirred using a magnetic stirring device.

After irradiation 0.37 g of Compound B (as described in example 3) are added and stirred in quickly using a stainless steel spatula. The homogenous formulation is applied on a strip of a polyethylene film (=strip A). A second polyethylene film, not coated with the adhesive, (=strip B) is pressed on strip A leaving no air between the layers. Immediately after laminating strip A and strip B, a flat wooden board is placed on top of the strips, which then is loaded with a 5 kg weight. After 5 h adhesion is tested by pulling the ends of the strips. The strips stick together when pulled at both ends.

### EXAMPLE 5

The procedure of Example 4 is repeated, instead of PLB-1 using 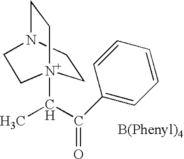
(PLB-2). The strips stick together when pulled at both ends.

### EXAMPLE 6

### Pigmented Coating Formulation

Formulation A is prepared by mixing the ingredients indicated below:
0.4% by weight of sensitizer isopropylthioxanthone (Darocur®ITX, provided by Ciba Specialty Chemicals)
59.6% by weight of butyl acetate as solvent
40.0% by weight of trimethylolpropane tris(3-mercapto-propionate) (provided by Aldrich)

Formulation B is prepared by mixing the ingredients indicated below:
9.0% by weight of transparent greenish-yellow organic pigment (Chromophtal® Yellow 8GN (provided by Ciba Specialty Chemicals)
76.0% by weight of bisphenol A epoxy resin (Bakelite EPR 162, provided by Bakelite AG)
8.0% by weight of polymeric dispersant (EFKA 4010, provided by Ciba Specialty Chemicals)
7.0% by weight of butyl acetate as solvent
   0.011 g of the photolatent base PLB-1 is dissolved in 0.4 g of Formulation A and the formulation is exposed for 10 minutes to UV light (fluorescent lamp Philips TL 40W/05 with main emission between 350 and 400 nm) in a 3.6 cm large crystallizer under vigorous stirring. 0.123 g of Formulation B is added and further stirred for a few seconds to get a homogeneous formulation.

The reaction of the coating is monitored at room temperature by ATR spectroscopy (Nicolet Magna-IR 750 spectrometer) to follow the SH (2564 cm⁻¹) and the epoxy (912 cm⁻¹) disappearance. The results are summarized in table 3

TABLE 3

| Time after irradiation | SH content (%) | Epoxy content (%) |
|---|---|---|
| Initial | 100 | 100 |
| 9 min | 79 | 75 |
| 15 min | 65 | 64 |

Further, the complete formulation, i.e. combined components A+B, is irradiated under the same conditions: the reaction is much more slower, as the results, collected in table 3a, show, but crosslinking is effected, too.

TABLE 3a

| Time after irradiation | SH content (%) | Epoxy content (%) |
|---|---|---|
| Initial | 100 | 100 |
| 10 min | 97 | 86 |
| 19 min | 93 | 80 |

### EXAMPLE 7

### Preparation of a Gel Coat Formulation

Formulation A is prepared by mixing the following ingredients:
0.4% by weight of sensitizer isopropylthioxanthone (Darocur®ITX, provided by Ciba Specialty Chemicals)
58.0% by weight of butyl acetate as solvent
42.0% by weight of trimethylolpropane tris(3-mercapto-propionate) (provided by Aldrich)

Formulation B is prepared as is described in example 4.

0.11 g of the of the photolatent base PLB-1 is dissolved in 4.3 g of formulation A and the formulation is exposed to UV light (fluorescent lamp Philips TL 40W/05 with main emission between 350 and 400 nm) for 20 minutes in a 6 cm large crystallizer under vigorous stirring. 1.43 g of Formulation B is added and further stirred for a few seconds to get a homogeneous formulation.

The reaction is monitored at room temperature by ATR spectroscopy (Nicolet Magna-IR 750 spectrometer) to follow the SH (2564 cm⁻¹) disappearance.

The results are collected in table 4.

TABLE 4

| Time after irradiation | SH content (%) |
|---|---|
| Initial | 100 |
| 50 min | 49 |

The gelcoat is tacky after 50 minutes at room temperature.

Further, the complete formulation, i.e. combined components A+B, is irradiated under the same conditions: the reaction, as the results, collected in table 4a, show is slower, nevertheless a crosslinking is effected.

TABLE 4a

| Time after irradiation | SH content (%) |
|---|---|
| Initial | 100 |
| 10 min | 100 |

In this case the gelcoat is still liquid after 50 minutes at room temperature.

### EXAMPLE 8

### Preparation of a Fiber Glass Composite

A formulation is prepared by mixing the following ingredients:
0.4% by weight of sensitizer isopropylthioxanthone (Darocur®ITX, provided by Ciba Specialty Chemicals)
59.0% by weight of butyl acetate as solvent
41.0% by weight of trimethylolpropane tris(3-mercapto-propionate) (provided by Aldrich)

0.33 g of the photolatent base PLB-1 is dissolved in 12.8 g of the formulation and the formulation is exposed to UV light (fluorescent lamp Philips TL 40W/05 with main emission between 350 and 400 nm) for 40 minutes in a 9 cm large crystallizer under vigorous stirring. 3.3 g of Bakelite EPR 162 (Bisphenol A epoxy resin) is added and further stirred for a few seconds to get an homogeneous formulation.

A glass fiber is soaked with the formulation, then another glass fiber is placed on top and again soaked with the formulation. The procedure is repeated until three glass fiber layers are soaked with the formulation.

The reaction is monitored at room temperature by ATR spectroscopy (Nicolet Magna-IR 750 spectrometer) to follow the SH (2564 cm⁻¹) disappearance.

The results are given in table 5.

TABLE 5

| Time after irradiation | SH content (%) |
|---|---|
| Initial | 100 |
| 36 min | 50 |
| 60 min | 50 |

The formulation is tack-free after 3 hours at room temperature.

Further, the complete formulation, i.e. combined formulations A+B, is irradiated under the same conditions: the reaction, as the results in table 5a show, is much more slower, however a crosslinking is effected.

TABLE 5a

| Time after irradiation | SH content (%) |
|---|---|
| Initial | 100 |
| 36 min | 73 |
| 60 min | 61 |

This formulation is still tacky after 3 h at room temperature

### EXAMPLE 9

### Curing of a NCO/OH Coating Formulation

Component A is prepared by mixing the following ingredients:
73.0% by weight of hydroxy bearing polyacrylate, 70% in butyl acetate (Desmophen A VP LS 2350, provided by Bayer AG
0.9% by weight of additive, 10% in butyl acetate (Byk 333, provided by Byk)
0.7% by weight of additive, 50% supply form (Byk 335, provided by Byk)
0.7% by weight of additive, 4% supply form (Byk 141, provided by Byk)
24.7% by weight of xylene/methoxypropylacetate/butylacetate as solvent in a ratio 1:1:1

Composition B is 100% by weight of aliphatic polyisocyanate, 90% in n-butyl acetate, (Desmodur N 3390 BA, provided by Bayer AG)

0.14 g of the photolatent base PLB-1 is dissolved in 3.76 g of Component A and the formulation is exposed to a 4.5 J/cm² UV dose (AETEK International) in a 1 cm large quartz cell. The formulation is further mixed with 1.03 g of Component B by means of a double feed spray gun and applied on a BaF₂ crystal.

The reaction is monitored at 130° C. by FTIR spectroscopy (Perkin Elmer 1600 FTIR spectrometer) to follow the NCO (2270 cm⁻¹) disappearance. The results are listed in table 6

TABLE 6

| Time at 130° C. after irradiation | NCO content (%) |
|---|---|
| Initial | 100 |
| 4 min | 71 |
| 15 min | 32 |

### EXAMPLE 10

### Curing of a NCO/OH Coating Formulation

0.14 g of the photolatent base PLB-1 is dissolved in 3.76 g of Component A, as described in example 9, and the formulation is exposed to a 4.5 J/cm² UV dose (AETEK International) in a 1 cm large quartz cell. The formulation is further mixed with 1.03 g of Component B, as described in example 9, by means of a double feed spray gun and applied in a dry thickness of about 40 μm on an aluminum panel. A fully cured, tack-free coating is obtained.

### EXAMPLE 11

### Curing of a NCO/OH Coating Formulation

The procedure according to example 10 is repeated, however instead of PLB-1 using PLB-2.

### EXAMPLE 12

The following formulation is prepared by mixing the respective ingredients:
56.16 parts of Joncryl 510 (acrylic polyol, provided by Johnson Polymer)
19.18 parts of Cymel 303 (hexamethoxymelamine, provided by Cytec)
14.16 parts of butyl alcohol
9.89 parts of methyl-pentyl ketone
0.61 parts of DC-57 (10% in methyl-pentyl ketone) levelling agent, provided by Dow Corning

2% of the photolatent acid α-(4-methylphenylsulfonyloxyimino)-4-thiomethylbenzyl cyamide) (PLA-1) are well dissolved in said formulation. The thus prepared coating formulation is then placed in a petri dish and irradiated on UV-curing equipment with 2×120 W/cm Hg-bulbs at a line speed of 5 m/min. After irradiation the coating formulation is applied on a white coil-coated aluminium panel with a dry film thickness of 50 microns. This panel is stoved for 30 minutes at 90° C. 2 hours after the panels are cooled down the pendulum hardness according to Konig (DIN 53157) is measured. The higher the pendulum hardness the better is the cure.

A second sample of the coating formulation is subjected to the same procedure, however without being pre-irradiated in the petri-dish.

Further, samples without a photolatent catalyst are subjected to the procedure as described above, i.e. with a pre-irradiation step and without.

The results are collected in the following table 7.

TABLE 7
Pendulum hardness in sec of the tack-free coatings

| | | pre-irradiation, |
|---|---|---|
| | curing in the dark, | followed by |
| Catalyst | without pre-irradiation | curing in the dark |
| None | tacky | tacky |
| photolatent acid | tacky | 31 |
| (PLA) | | |

### EXAMPLE 13

### Cationic UV-curable Adhesive

Component A is prepared by mixing the ingredients indicated below until homogenous:
20.0% by weight of polyester polyol (Tone Polyol 0310, provided by DOW Chemical)
5.0% by weight of butyl acetate as solvent
4.0% by weight of a cationic photoinitiator 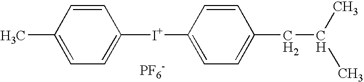
(Irgacure® 250, provided by Ciba Specialty Chemicals—=PLA-2)

Component B: cycloaliphatic epoxide resin (Cyracure Resin UVR 6105, provided by DOW Chemical)

Component A is applied on a polycarbonate strip (2 cm×12 cm, thickness approx. 1 mm) using a wire coater (wet film thickness 4 μm) and placed under a UV-lamp (Hoenle UVASPOT, Hg-bulb) and irradiated for 10 min (Strip A).

Component B is applied on a polycarbonate strip (2 cm×12 cm, thickness approx. 1 mm) using a wire coater (wet film thickness 12 μm) (Strip B).

After irradiation, Strip A and B are pressed together on the coated sides leaving no air between the layers. Immediately after laminating Strip A and Strip B, a flat wooden board is placed on top of the strips, which then is loaded with a 5 kg weight. After 5 h adhesion is tested by pulling the ends of the strips. The strips stick together when pulled at both ends.

### EXAMPLE 14

### Preparation of a Solventless Epoxy Adhesive

The formulation is prepared by mixing the ingredients indicated below:
97.0% by weight of bisphenol-A/bisphenol-F based epoxy resin (Bakelite EPR 144, provided by Bakelite)
3.0% by weight of the photolatent base (PLB-2)

0.5 g of the formulation are placed in an open clear glass crystallizer (layer thickness approx. 1 mm) and exposed to a 4.5 J/cm² UV dose (AETEK international).

After irradiation, the formulation is applied on a strip of a polyethylene film (=strip A). A second polyethylene film, not coated with the adhesive, (=strip B) is pressed on strip A leaving no air between the layers. Immediately after laminating strip A and strip B, a flat wooden board is placed on top of the strips, which then is loaded with a 5 kg weight. Adhesion is tested by pulling the ends of the strips. The strips stick together when pulled at both ends.

### EXAMPLE 15

### Preparation of an Epoxy Coating

The formulation is prepared by mixing the ingredients indicated below:
80.0% by weight of diglycidylether of bisphenol-A (Bakelite EPR 162, provided by Bakelite)
17.0% by weight of butyl acetate
3.0% by weight of the photolatent base 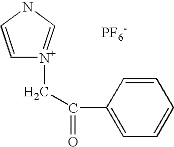
(PLB-3)

0.5 g of the formulation are placed in an open clear glass crystallizer (layer thickness approx. 1 mm) and exposed to a 4.5 J/cm² UV dose (AETEK international).

After irradiation, a 40 μm thick formulation layer is applied on an coil coated aluminium panel by means of a wire coater. A fully cured, tack-free coating is obtained.

### EXAMPLE 16

### Preparation of a Polyurethane Foam

Polyether polyol (Lupranol® 2080, trifunctional polyether polyol having primary hydroxyl groups; hydroxyl number 48 mg KOH/g, water content less than 0.1%, acid number less than 0.1 mg KOH/g, containing 0.45% of the stablizer Irgastab® PUR 55), the photolatent base PLB-1 and isopropylthioxanthone (Darocur ITX) are mixed in a ratio 100:5:0.5. 10 g of said mixture is exposed to UV light (fluorescent lamp Philips TL 40W/05 with main emission between 350 and 400 nm) four times for 5 minutes.

7.5 g of the irradiated solution is subsequently dissolved in additional 78.60 g of Lupranol 2080. 4.96 g of a solution consisting of 0.96 Tegostab® BF 2370 (supplied by Goldschmidt, Germany) and 4 g of deionized water are subsequently added and the reaction mixture is stirred vigorously for 10 seconds at 2600 rpm. 1.6 g of a solution of Kosmos 29 (supplied by Goldschmidt, Germany)/Lupranol 2080 (ratio 1:9) is then added and the reaction mixture is again stirred vigorously for 18 seconds at 2600 rpm.

48.88 g of an isocyanate (Lupranat® T80, supplied by BASF; toluoylene-2,4- and toluoylene-2,6-diisocyanate mixture) is then added with continuous stirring for 5 to 7 seconds at 2600 rpm. The mixture is then poured into a 20×20×20 cm cake-box and the foaming reaction starts, resulting in the building of a foam block.

1. Process for the application of a photolatent catalyst (a)
wherein a composition of matter, comprising said catalyst, is subjected to irradiation before being further processed, characterized in that the photolatent catalyst is (a1) a photolatent acid of the formula VI 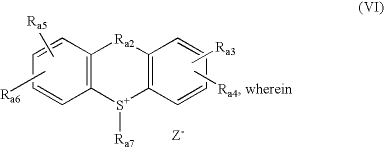
Rₐ₂ is a direct bond, S, O, CH₂, (CH₂)₂, CO or NR₉₆;
Rₐ₃, Rₐ₄, Rₐ₅ and Rₐ₆ independently of one another are H, C₁-C₂₀alkyl, C₃-C₈cycloalkyl, C₁-C₂₀alkoxy, C₂-C₂₀alkenyl, CN, OH, halogen, C₁-C₆alkylthio, phenyl, naphthyl, phenyl-C₁-C₇alkyl, naphtyl-C₁-C₃alkyl, phenoxy, naphthyloxy, phenyl-C₁-C₇alkyloxy, naphtyl-C₁-C₃alkyloxy, phenyl-C₂-C₆alkenyl, naphthyl-C₂-C₄alkenyl, S-phenyl, (CO)Rₐ₈, O(CO)Rₐ₈, (CO)ORₐ₈, SO₂Rₐ₈, OSO₂Rₐ₈;
Rₐ₇ is C₁-C₂₀alkyl, C₁-C₂₀hydroxyalkyl, 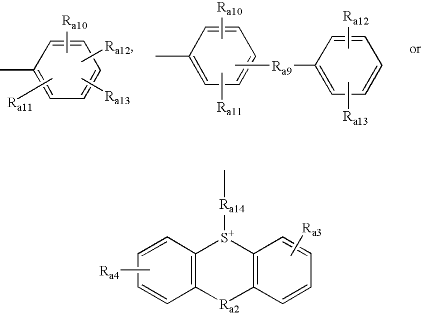
Rₐ₈ is H, C₁-C₁₂alkyl, C₁-C₁₂hydroxyalkyl, phenyl, naphthyl or biphenylyl;
Rₐ₉ is a direct bond, S, O or CH₂;
Rₐ₁₀, Rₐ₁₁, Rₐ₁₂ and Rₐ₁₃ independently of one another have one of the meanings as given for Rₐ₃; or
Rₐ₁₀ and Rₐ₁₂ are joined to form a fused ring system with the benzene rings to which they are attached;
Rₐ₁₄ is 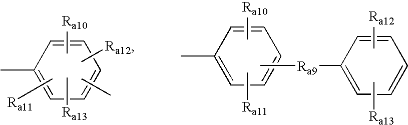
Z is an anion or wherein the photolatent acid (a1) is a compound selected from the group consisting of aromatic phosphonium salts, aromatic iodonium salts or oxime-based photolatent acids; or the photolatent catalyst (a) is (a2) a photolatent base compound, provided that (3,4-dimethoxy-benzoyl)-1-benzyl-1-dimethylamino propane is excluded, if the composition of matter comprises isocyanates in combination with thiols.

2. Process according to claim 1, wherein
(A) the photolatent catalyst (a) is a photolatent acid (a1) and the composition of matter comprises acid-catalysed curable compounds (b); or wherein
(B) the photolatent catalyst (a) is a photolatent base (a2) and the composition of matter comprises base-catalysed curable compounds (c); or wherein
(C) the photolatent catalyst (a) is a mixture of at least one photolatent base catalyst (a2) and at least one photolatent acid catalyst (a1) and wherein the composition of matter comprises a mixture of acid-catalysed curable compounds (b) and base-catalysed curable compounds (c), provided that (a1) and (a2) are selectively activated.

3. Process according to claim 1, wherein the composition of matter comprising the photocatalyst additionally comprises a dye or pigment (g).

4. A process for the application of a photolatent catalyst (a) according to claim 1, wherein a composition of matter, comprising said catalyst, is subjected to irradiation before being further processed,
wherein the composition of matter is a laquer formulation, comprising a polyol in combination with an isocyanate and as photolatent catalyst a photolatent base (a2) of the formula VIII, VIIIa and VIIIb 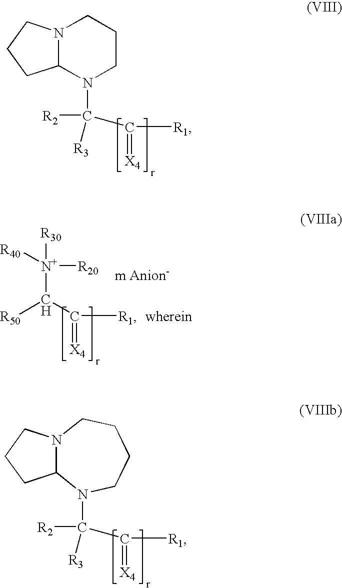
r is 0 or 1;
X₄ is CH₂ or O;
R₂ and R₃ are each independently of the other hydrogen or C₁-C₂₀alkyl;
R₁ is unsubstituted or C₁-C₁₂alkyl- or C₁-C₁₂alkoxy-substituted phenyl, naphthyl or biphenylyl;
R₂₀, R₃₀ and R₄₀, together with the linking nitrogen atom, are a group of the structural formula (b) or (c); 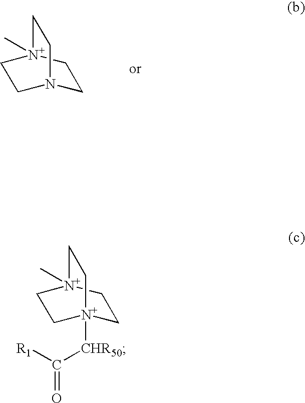
Anion is any anion capable to form the salt; and
m is the number of positively charged N-atoms in the molecule.

5. A process for the application of a photolatent catalyst (a) according to claim 1, wherein a composition of matter, comprising said catalyst, is subjected to irradiation before being further processed, wherein the composition of matter is a laquer formulation, comprising an epoxide component and as photolatent catalyst a photolatent base (a2) of the formula VIIIa, 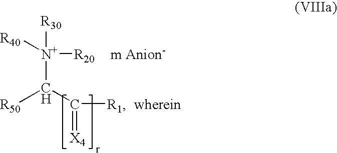
r is 0 or 1;
X₄ is CH₂ or O;
R₁ is unsubstituted or C₁-C₁₂alkyl- or C₁-C₁₂alkoxy-substituted phenyl, naphthyl or biphenylyl;
R₂₀, R₃₀ and R₄₀, together with the linking nitrogen atom, are a group of the structural formula (a), (b) or (c); 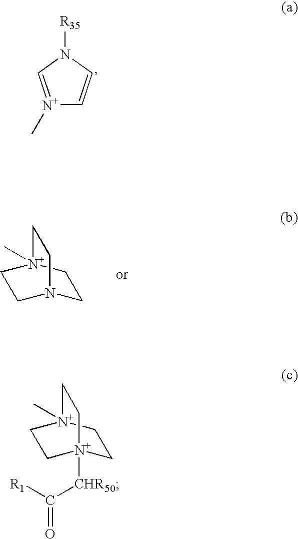
R₃₅ is hydrogen or C₁-C₁₈alkyl;
Anion is any anion capable to form the salt; and
m is the number of positively charged N-atoms in the molecule.

6. A process for the application of a photolatent catalyst (a), according to claim 1, wherein the composition of matter is an adhesive.

7. A process according to claim 6, wherein the photolatent catalyst is a photolatent base (a2) and the composition of matter is a base-catalysed curable compound (c).

8. Process according to claim 1, wherein the further processing involves the application of the irradiated composition of matter to a substrate, optionally followed by further mechanical processing steps of the coated substrate; the preparation of a foam; the preparation of a polymer; the preparation of a fiber; the preparation of a gelcoat; the preparation of a composite, the preparation of an adhesive, the preparation of a clear coating or a pigmented coating, a printing ink, an inkjet ink, or the preparation of a coating that has an additional material incorporated.

9. Process according to claim 1, which is repeatedly applied, the photocatalyst in each repetition step being the same or different from the other steps and independently being either a photolatent acid and/or a photolatent base.

10. Process according to claim 1, wherein the composition of matter is irradiated directly in the storage tank and subsequently subjected to the further processing.

11. A process according to claim 1, wherein the further processing is an additional curing step using UV-light and/or heat.

12. Substrate covered with a composition of matter according to the process of claim 1.

13. A process for the application of a photolatent catalyst (a)
wherein a composition of matter, comprising said catalyst, is subjected to irradiation before being further processed, characterized in that the further processing resides in the preparation of a foam and the composition of matter comprises polyol and isocyante components and as photolatent catalyst a photolatent base (a2).

14. A process according to claim 1, wherein Z is PF₆, SbF₆, AsF₆, BF₄, (C₆F₅)₄B, Cl, Br, HSO₄, CF₃—SO₃, F—SO₃, 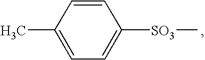
CH₃—SO₃, ClO₄, PO₄, NO₃, SO₄, CH₃—SO₄ or

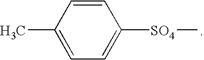

15. Process according to claim 14, wherein
(D) the photolatent catalyst (a) is a photolatent acid (a1) and the composition of matter comprises acid-catalysed curable compounds (b); or wherein
(E) the photolatent catalyst (a) is a mixture of at least one photolatent base catalyst (a2) and at least one photolatent acid catalyst (a1) and wherein the composition of matter comprises a mixture of acid-catalysed curable compounds (b) and base-catalysed curable compounds (c), provided that (a1) and (a2) are selectively activated.

16. Process according to claim 14, wherein the composition of matter comprising the photocatalyst additionally comprises a dye or pigment (g).

17. A process for the application of a photolatent catalyst (a), according to claim 14, wherein the composition of matter is an adhesive.

18. Process according to claim 14, wherein the composition of matter is irradiated directly in the storage tank and subsequently subjected to the further processing.

19. A process according to claim 14, wherein the further processing is an additional curing step using UV-light and/or heat.
